(19) United States
(12) Patent Application Publication
(10) Pub. No.: US 2008/0318938 A1
(43) Pub. Date: Dec. 25, 2008

(54) METHODS FOR TREATING ABERRANT CELL PROLIFERATION DISORDERS

(76) Inventors: David M. RYCKMAN, San Diego, CA (US); Denis Drygin, San Diego, CA (US); Jeffrey P. Whitten, Santee, CA (US); Kenna Anderes, San Diego, CA (US); Katy Trent, San Diego, CA (US); Levan Darjania, Escondido, CA (US); Mustapha Haddach, San Diego, CA (US); Sean O'Brien, Carlsbad, CA (US); William G. Rice, Del Mar, CA (US)

Correspondence Address: MORRISON & FOERSTER LLP, 12531 HIGH BLUFF DRIVE, SUITE 100, SAN DIEGO, CA, 92130-2040, US

(21) Appl. No.: 12/143,223

(22) Filed: Jun. 20, 2008

Related U.S. Application Data
(60) Provisional application No. 60/945,870, filed on Jun. 22, 2007.
(60) Provisional application No. 61/023,695, filed on Jan. 25, 2008.
(60) Provisional application No. 61/050,155, filed on May 2, 2008.

(51) Int. Cl. A61K 31/5383; A61P 35/02; A61P 35/00

(52) U.S. Cl. 514/229.5

(57) ABSTRACT

The invention relates in part to methods for inhibiting proliferation of cancer cells and treating one or more cell proliferative conditions using compounds described herein.

### RELATED APPLICATIONS

This application claims benefit of priority to U.S. Provisional Application Ser. No. 60/945,870 filed 22 Jun. 2007; U.S. Provisional Application Ser. No. 61/023,695 filed 25 Jan. 2008; and U.S. Provisional Application Ser. No. 61/050,155 filed 2 May 2008. The contents of these documents are incorporated herein by reference in their entirety.

### FIELD OF THE INVENTION

The invention relates in part to methods for treating biological disorders involving aberrant cell proliferation.

### DISCLOSURE OF THE INVENTION

The present invention in part provides chemical compounds having certain biological activities that include, but are not limited to, inhibiting cell proliferation. It has been determined that compounds described herein inhibit proliferation of cells involved in conditions associated with aberrant cell proliferation. Such conditions include cancers and inflammation conditions.

Compounds of the invention fall within the general formulae described hereafter. Preferred therapeutic agents include compounds having the general formulae TA1-1A, TA1-1B, and TA4-1A, in certain embodiments. For example, therapeutic agent TA1-1B is useful for inhibiting proliferation of cancer cells, including, but not limited to, leukemia cells, lymphoma cells, breast cancer cells, lung cancer cells (e.g., small cell or non-small cell lung cancer cells), central nervous system cancer cells (e.g., brain cancer cells), skin cancer cells (e.g., melanoma cells), ovarian cancer cells, prostate cancer cells, renal cancer cells (e.g., kidney cancer cells) and colorectal cancer cells. TA1-1B is also useful for inhibiting proliferation of liver cancer cells, pancreatic cancer cells, adrenal gland cancer cells, thymic cancer cells, lymph node cancer cells, stomach cancer cells, appendix cancer cells, small bowel cancer cells, head and neck cancer cells, heart cancer cells, pituitary gland cancer cells, parathyroid gland cancer cells, and thyroid gland cancer cells.

Compound TA1-1B advantageously crosses the blood-brain barrier and can be useful for inhibiting proliferation of brain cancer cells.

Compound TA4-1A advantageously crosses the blood-brain barrier and can be useful for inhibiting proliferation of brain cancer cells.

TA1-1A and TA1-1B are useful for inhibiting proliferation of tumors overexpressing peptide receptors, including, but not limited to, neuroendocrine tumors, paragangliomas, pheochromocytomas, small-cell lung cancers, medullary thyroid cancers, breast cancers, renal cell cancers, malignant lymphomas, GIST, GEP NET, sex cord stromal ovarian cancers, medulloblastomas, gliomas, exocrine pancreatic cancers, meningiomas, Ewing sarcomas, adrenal cancers, insulinomas, gastrinomas, nonfunctioning pituitary adenomas, ileal carcinoids, glucagonomas, VIPomas, GH-producing pituitary adenomas, gut carcinoids, astrocytomas, leiomyomas, and bronchial carcinoids. Accordingly, these compounds are useful for treating subjects having these cancers.

Certain compositions comprise a compound described herein in combination with a cell. The cell may be from a cell line, such as a cancer cell line. In the latter embodiments, the cancer cell line is sometimes a breast cancer, prostate cancer, pancreatic cancer, lung cancer, hemopoietic cancer (e.g. leukemia), colorectal cancer, skin cancer, ovary cancer cell line, and can be any cell line described herein.

These and other embodiments of the invention are described in the description that follows.

### BRIEF DESCRIPTION OF THE DRAWING

FIG. 1A and FIG. 1B show effects of a representative compound on RNA synthesis and cell viability, respectively, in bone marrow cells.

FIG. 2 shows the distribution of TA1-1B in rat plasma, blood and brain as a function of time post-dose. TA1-1B is administered QDx1 and the concentration (μM) is determined 2 hr post dose; or QDx5, and the concentration (μM) is determined 2 hr post dose (98 hr), 24 hour post-dose (122 hr) and 48 hr post-dose (146 hr).

FIG. 3 shows the distribution of TA1-1B in rat adrenal glands and bone marrow as a function of time post-dose. TA1-1B is administered QDx1 and the concentration (μM) is determined 2 hr post dose; or QDx5, and the concentration (μM) is determined 2 hr post dose (98 hr), 24 hour post-dose (122 hr) and 48 hr post-dose (146 hr).

### EMBODIMENTS OF THE INVENTION

Therapeutic agents described herein are useful to inhibiting cell proliferation and can be utilized to treat conditions associated with aberrant cell proliferation, such as certain cancers and inflammation conditions. The therapeutic agents can result in cell apoptosis and cell necrosis, and can specifically target proliferation of cells leading to an aberrant cell proliferation condition over “normal” cells. Examples of therapeutic agents and conditions that can be treated by the agents are described hereafter.

Therapeutic Agents

The invention provides methods to treat conditions associated with aberrant cell proliferation, such as cancers and inflammation conditions, by administering to a subject in need of such treatment a therapeutically effective amount of a therapeutic agent in an amount effective to treat the condition.

The therapeutic agent may be administered in combination with another agent, and the combination may be administered as separate pharmaceutical compositions or admixed in a single pharmaceutical composition. The therapeutic agent and the combination agent also may be administered separately, including at different times and with different frequencies, as long as the combination agent is administered at a time that increases the potency of the therapeutic agent. In some embodiments, the combination agent and the therapeutic agent are administered at the same time, whether in separate dosages or admixed in a single dosage. Where the frequency of administration of the two materials can be adjusted to match, the combination agent and therapeutic agent are preferably combined into a single pharmaceutical composition, so the treated patient may receive a single dosage (e.g., oral or injection), for example.

Therapeutic agents of the invention are compounds that inhibit cell proliferation. Certain therapeutic agents can inhibit RNA biosynthesis, and some can bind to certain motifs in nucleic acids. Therapeutic agents to be used can be selected from several different classes of compounds, such as those described below. The therapeutic agents are useful for the treatment of cancer and other indications such as inflammatory disorders, and methods for making and using them are known in the art. Several preferred classes of these therapeutic agents are described below.

In one aspect, the therapeutic agent can be a compound of formula (TA1-1):

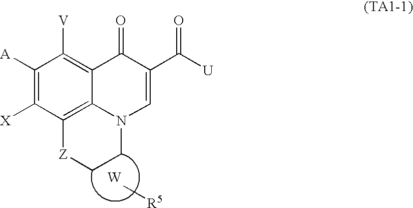

and pharmaceutically acceptable salts, esters and prodrugs thereof;

wherein V is H, halo, or NR¹R²;

A is H, fluoro, or NR¹₂;

Z is O, S, NR¹ or CH₂;

U is OR² or NR¹R²;

X is OR², NR¹R², halo, azido, or SR²;

n is 1-3;

wherein in NR¹R², R¹ and R² may form a double bond or a ring, each of which is optionally substituted;

R¹ is H or a C₁₋₆ alkyl;

R² is H or a C₁₋₁₀ alkyl or C₂₋₁₀ alkenyl optionally containing one or more non-adjacent heteroatoms selected from N, O, and S, and optionally substituted with an optionally substituted carbocyclic or heterocyclic ring; or R² is an optionally substituted heterocyclic ring, aryl or heteroaryl;

R⁵ is a substituent at any position on W; and is H, OR², C₁₋₆ alkyl, C₂₋₆ alkenyl, each optionally substituted by halo, ═O or one or more heteroatoms; or R⁵ is an inorganic substituent; and

W is an optionally substituted aryl or heteroaryl, which may be monocyclic or fused with a single or multiple ring and optionally containing a heteroatom;

or a compound having formula (TA1-2):

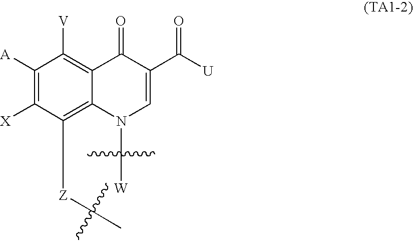

and pharmaceutically acceptable salts, esters and prodrugs thereof;

wherein V, A, X, Z and U are as defined in formula TA1-1, and W is selected from the group consisting of

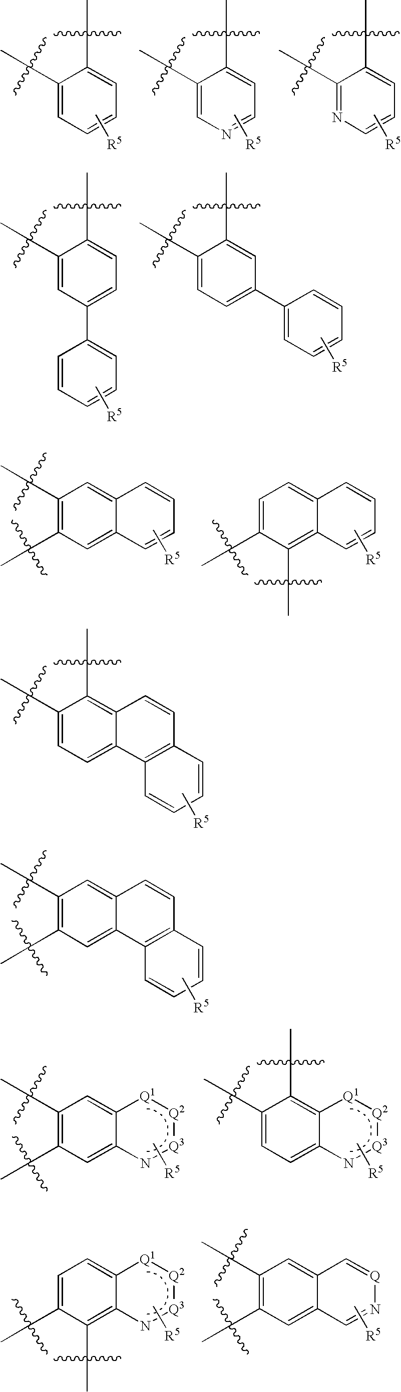

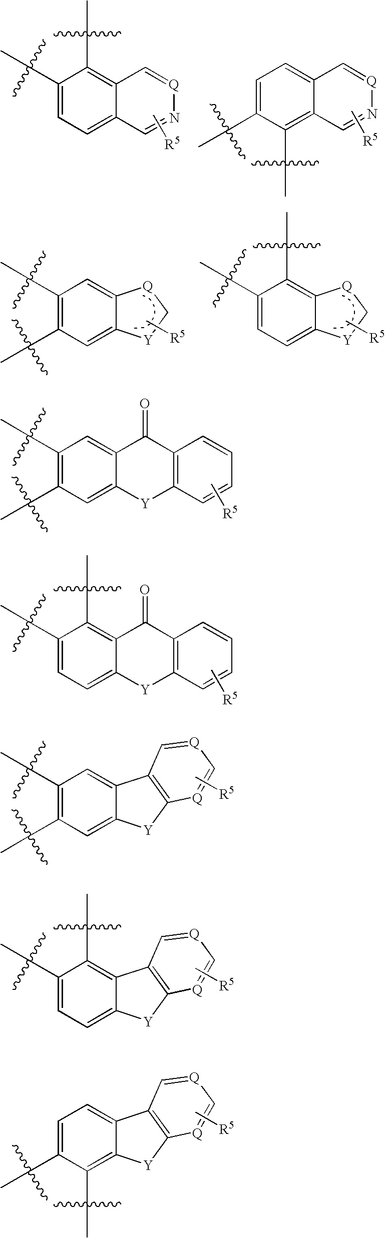

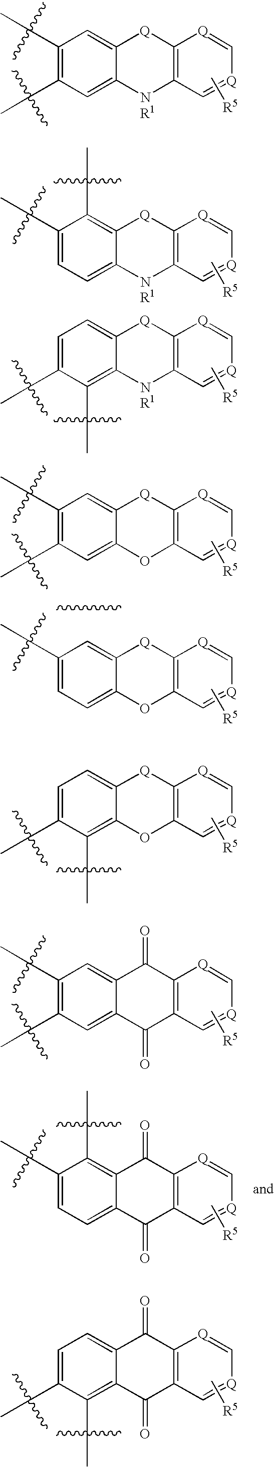

wherein Q, Q¹, Q², and Q³ are independently CH or N;

Y is independently O, CH, ═O or NR¹; and

R⁵ is as defined in formula 1.

Compounds of this structure, and methods for making and using them, are described in U.S. patent application Ser. No. 11/106,909, naming Whitten, et al., which is entitled SUBSTITUTED QUINOBENZOXAZINE ANALOGS AND METHODS OF USING THEREOF, and was filed on Apr. 15, 2005.

In a specific embodiment of the therapeutic agents of formula (TA1-1), the therapeutic agent is a compound having a structure of formula (TA1-1A):

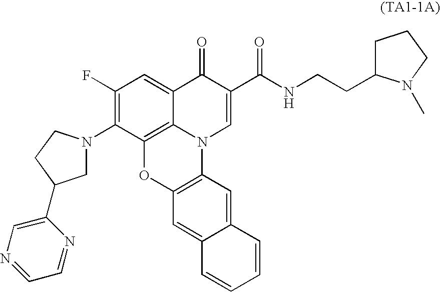

or a pharmaceutically acceptable salt, ester or prodrug thereof, or a specific isomer or mixture of isomers thereof.

In a specific embodiment of the therapeutic agents of formula (TA1-1), the therapeutic agent is a compound having a structure of formula (TA1-1B):

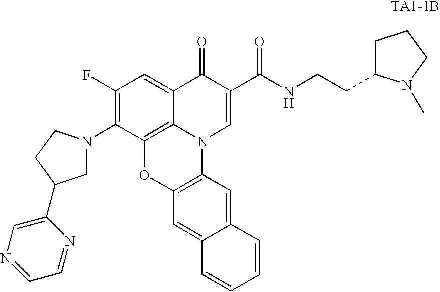

or a pharmaceutically acceptable salt, ester or prodrug thereof, or a specific isomer or mixture of isomers thereof. Note that TA1-1A is a mixture of four stereoisomers and TA1-1B is a mixture of two of those four stereoisomers. In limited testing, TA1-1A and TA1-1B were found to have similar activity, but the data presented herein were generated with the isomer mixture corresponding to formula TA1-1B.

In another aspect, the therapeutic agent can be a compound having the general formula:

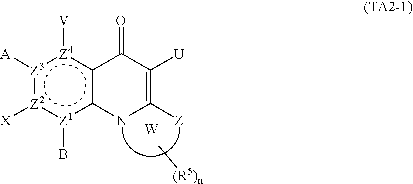

and pharmaceutically acceptable salts, esters and prodrugs thereof;

wherein B, X, A, or V is absent if Z¹, Z², Z³, or Z⁴ respectively is N, and independently H, halo, azido, R², CH₂R², SR², OR² or NR¹R² if Z¹, Z², Z³, or Z⁴ respectively is C; or

A and V, A and X, or X and B may form a carbocyclic ring, heterocyclic ring, aryl or heteroaryl, each of which may be optionally substituted and/or fused with a cyclic ring;

Z is O, S, NR¹, CH₂, or C═O;

Z¹, Z², Z³ and Z⁴ are C or N, provided any three N are non-adjacent;

W together with N and Z forms an optionally substituted 5- or 6-membered ring that is fused to an optionally substituted saturated or unsaturated ring; said saturated or unsaturated ring may contain a heteroatom and is monocyclic or fused with a single or multiple carbocyclic or heterocyclic rings;

U is SO₃R², SO₂NR¹R², SO₂NR¹NR¹R², SO₂NR¹OR², SO₂NR¹—(CR¹₂)ₙ—NR³R⁴ or SO₂NR¹NR¹—(CR¹₂)ₙ—NR³R⁴ or SO₂NR¹—O—(CR¹₂)ₙ—NR³R;

in each NR¹R², R¹ and R² together with N may form an optionally substituted ring;

in NR³R⁴, R³ and R⁴ together with N may form an optionally substituted ring;
R¹ and R³ are independently H or C₁₋₆ alkyl;
each R² is H, or a C₁₋₁₀ alkyl or C₂₋₁₀ alkenyl each optionally substituted with a halogen, one or more non-adjacent heteroatoms, a carbocyclic ring, a heterocyclic ring, an aryl or heteroaryl, wherein each ring is optionally substituted; or R² is an optionally substituted carbocyclic ring, heterocyclic ring, aryl or heteroaryl;

R⁴ is H, a C₁₋₁₀ alkyl or C₂₋₁₀ alkenyl optionally containing one or more non-adjacent heteroatoms selected from N, O and S, and optionally substituted with a carbocyclic or heterocyclic ring; or R³ and R⁴ together with N may form an optionally substituted ring;

each R⁵ is a substituent at any position on ring W; and is H, OR², amino, alkoxy, amido, halogen, cyano or an inorganic substituent; or R⁵ is C₁₋₆ alkyl, C₂₋₆ alkenyl, C₂₋₆ alkynyl, —CONHR¹, each optionally substituted by halo, carbonyl or one or more non-adjacent heteroatoms; or two adjacent R⁵ are linked to obtain a 5-6 membered optionally substituted carbocyclic or heterocyclic ring that may be fused to an additional optionally substituted carbocyclic or heterocyclic ring; and

n is 1-6.

In the above formula (TA2-1), B may be absent when Z¹ is N, or is H or a halogen when Z¹ is C.

In yet another embodiment, a therapeutic agent can have a structure of general formula (TA2-2) or (TA2-3):

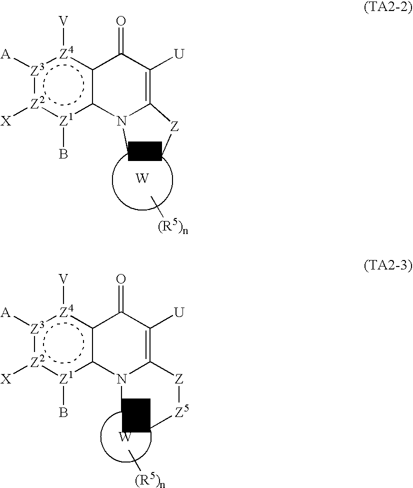

and pharmaceutically acceptable salts, esters and prodrugs thereof;

wherein V, A, X, B, W, U, Z, Z¹, Z², Z³, Z⁴ and n are as described above;

Z⁵ is O, NR¹, CR⁶, or C═O;

R⁶ is H, C₁₋₆ alkyl, hydroxyl, alkoxy, halo, amino or amido; and

Z and Z⁵ may optionally form a double bond.

In yet another embodiment, a therapeutic agent can have a structure of general formula (TA3-1) or (TA3-2):

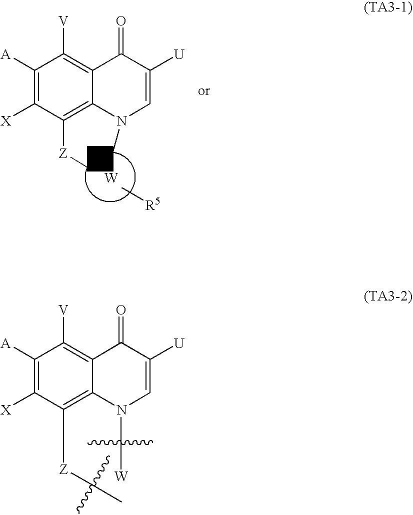

and pharmaceutically acceptable salts, esters and prodrugs thereof, where V, A, X, Z, W, U and R⁵ are previously described with respect to formulae TA2-1, TA2-2 and TA2-3.

In certain embodiments pertaining to compounds having structures of formulae TA2-1, TA2-2, TA2-3, TA3-1 and TA3-2, W together with N and Z in the above formula forms an optionally substituted 5- or 6-membered ring that is fused to an optionally substituted aryl or heteroaryl selected from the group consisting of:

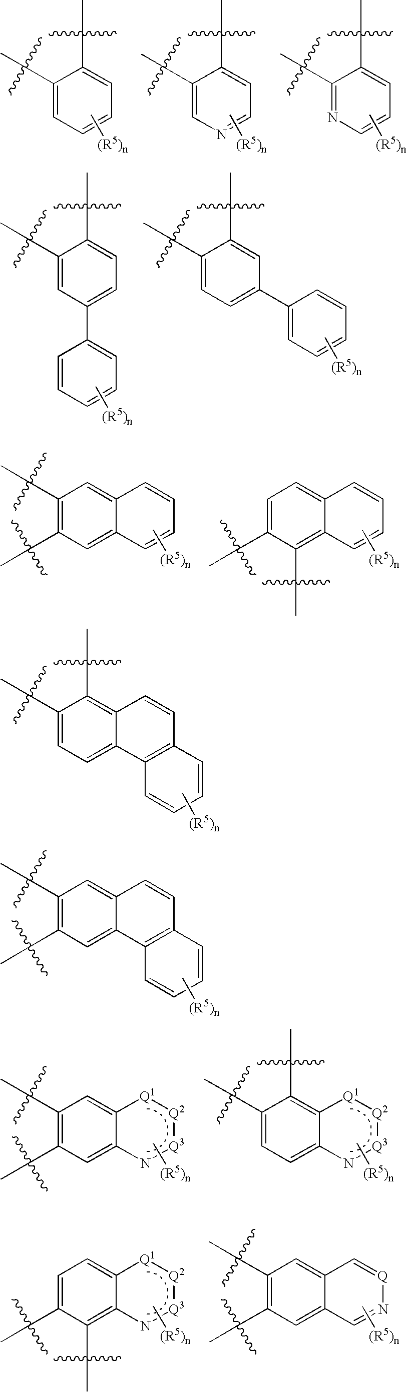

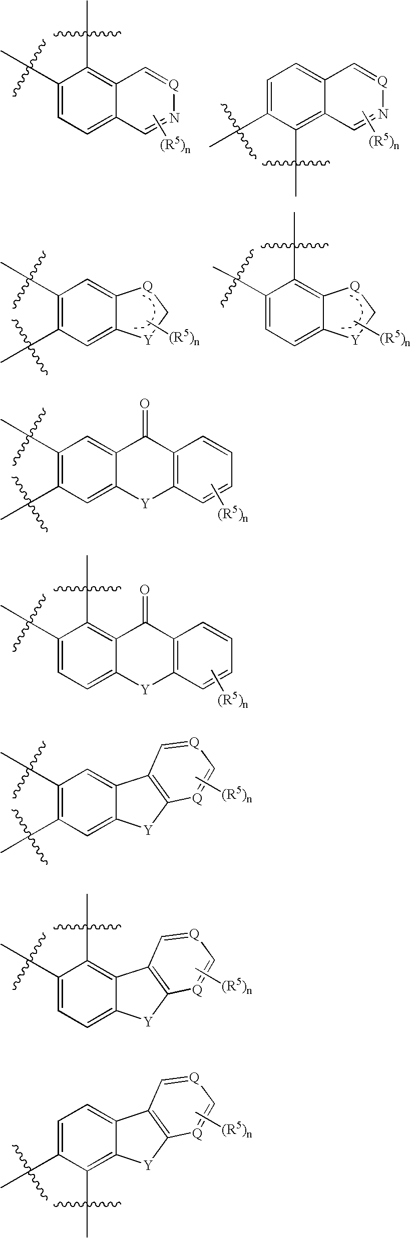

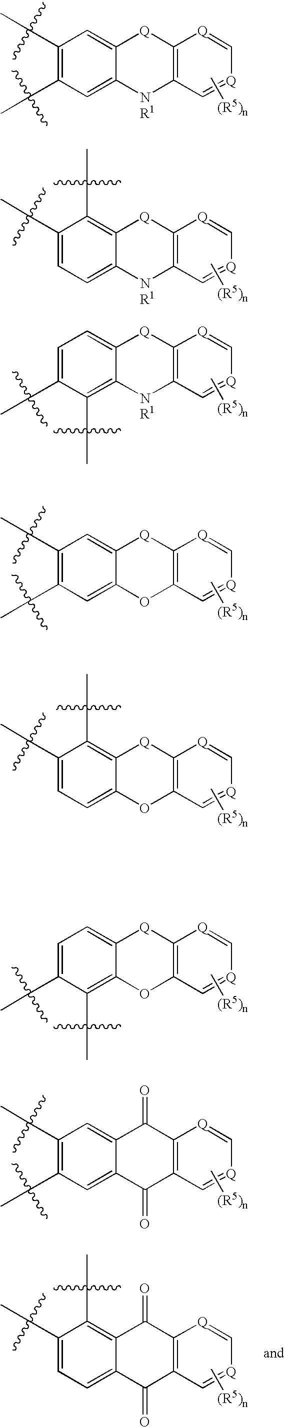

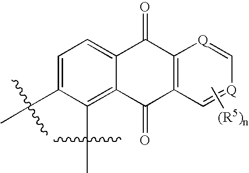

wherein each Q, Q¹, Q², and Q³ is independently CH or N;

Y is independently O, CH, C═O or NR¹;

n and R⁵ are as defined above.

In certain embodiments pertaining to compounds having structures of formulae TA2-1, TA2-2, TA2-3, TA3-1 and TA3-2, W together with N and Z may form a group having the formula selected from the group consisting of

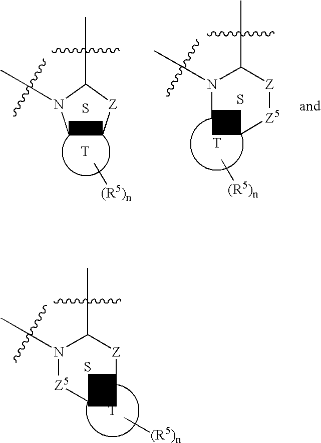

wherein Z is O, S, CR¹, NR¹, or C═O;
each Z⁵ is C(R⁶)₂, NR¹, or C═O, or Z and Z⁵ if adjacent can be —CR⁶═CR⁶— or —CR⁶═N—, and provided Z and Z⁵ if adjacent are not both NR¹;
each R¹ is H, C₁₋₆ alkyl, COR² or S(O)ₚR² wherein p is 1-2;
each R is independently H, or a substituent known in the art, including but not limited to hydroxyl, alkyl, alkoxy, halo, amino, or amido; and
ring S and ring T may be saturated or unsaturated.

In some embodiments pertaining to compounds having structures of formulae TA2- 1, TA2-2, TA2-3, TA3-1 and TA3-2, W together with N and Z forms a 5- or 6-membered ring that is fused to a phenyl.

In certain embodiments pertaining to compounds having structures of formulae TA2-1, TA2-2, TA2-3, TA3-1 and TA3-2, U may be SO₂NR¹R² or SO₂NR¹OR² or SO₂NR¹NR¹R², wherein R¹ is H, and R² is a C₁₋₁₀ alkyl optionally substituted with a heteroatom, a C₃₋₆ cycloalkyl, aryl or a 5-14 membered heterocyclic ring containing one or more N, O or S as ring members. For example, R² may be a C₁₋₁₀ alkyl substituted with an optionally substituted morpholine, thiomorpholine, imidazole, aminodithiadazole, pyrrolidine, piperazine, pyridine or piperidine. In other examples, R¹ and R² together with N form an optionally substituted piperidine, pyrrolidine, piperazine, morpholine, thiomorpholine, imidazole, or aminodithiazole. In some embodiments, U is SO₂NR¹R², and in some of these embodiments R¹ is H.

In some embodiments pertaining to compounds having structures of formulae TA2-1, TA2-2, TA2-3, TA3-1 and TA3-2, U may be SO₂NR¹—(CR¹₂)ₙ—NR³R⁴ or SO₂NR¹NR¹—(CR¹₂)ₙ—NR³R⁴ or SO₂NR¹O—(CR¹₂)ₙ—NR³R⁴; n is 1-4; and R³ and R⁴ in NR³R⁴ together form an optionally substituted piperidine, pyrrolidine, piperazine, morpholine, thiomorpholine, imidazole, or aminodithiazole. In some examples, U is SO₂NH—(CH₂)ₙ—NR³R⁴ wherein R³ and R⁴ together with N form an optionally substituted pyrrolidine, which may be linked to (CH₂)ₙ at any position in the pyrrolidine ring. In some embodiments, U is SO₂NR¹—(CR¹₂)ₙ—NR³R⁴, and in some of these embodiments R¹ is H. In one embodiment, R³ and R⁴ together with N form an N-methyl substituted pyrrolidine.

The preparation and activity of these compounds of formula (TA3-1) are described in International Patent Application No. PCT/US07/70794, filed Jun. 8, 2007, naming Nagasawa, et al., and entitled QUINOLONE ANALOGS DERIVATIZED WITH SULFONIC ACID, SULFONATE OR SULFONAMIDE.

In another aspect, the therapeutic agent is a compound of the following formula:

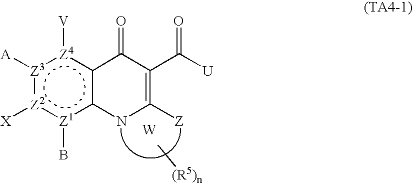

and pharmaceutically acceptable salts, esters and prodrugs thereof;

wherein B, X, A, or V is absent if Z², Z³, or Z⁴, respectively, is N, and independently H, halo, azido, R², CH₂R², SR², OR² or NR¹R² if Z², Z³, or Z⁴, respectively, is C; or

A and V, A and X, or X and B may form a carbocyclic ring, heterocyclic ring, aryl or heteroaryl, each of which may be optionally substituted and/or fused with a cyclic ring;

Z is O, S, NR¹, CH₂, or C═O;

Z¹, Z², Z³ and Z⁴ are C or N, provided any three N are non-adjacent;

W together with N and Z forms an optionally substituted 5- or 6-membered ring that is fused to an optionally substituted saturated or unsaturated ring; said saturated or unsaturated ring may contain a heteroatom and is monocyclic or fused with a single or multiple carbocyclic or heterocyclic rings;

U is R², OR², NR¹R², NR¹—(CR¹₂)ₙ—NR³R⁴, or N═CR¹R², wherein in N═CR¹R²R¹ and R² together with C may form a ring,

provided U is not H, and when U is OH, OR² or NH₂, then at least one of Z¹-Z⁴ is N;

in each NR¹R², R¹ and R² together with N may form an optionally substituted ring;

in NR³R⁴, R³ and R⁴ together with N may form an optionally substituted ring;
R¹ and R³ are independently H or C₁₋₆ alkyl;
each R² is H, or a C₁₋₁₀ alkyl or C₂₋₁₀ alkenyl each optionally substituted with a halogen, one or more non-adjacent heteroatoms, a carbocyclic ring or a heterocyclic ring, wherein each ring is aryl or heteroaryl and optionally substituted; or R is an optionally substituted carbocyclic ring, heterocyclic ring, aryl or heteroaryl;

R⁴is H, a C₁₋₁₀ alkyl or C₂₋₁₀ alkenyl optionally containing one or more non-adjacent heteroatoms selected from N, O and S, and optionally substituted with an optionally substituted carbocyclic or heterocyclic ring; or R³ and R⁴ together with N may form an optionally substituted ring;

each R⁵is a substituent at any position on ring W; and is H, OR², amino, alkoxy, amido, halogen, cyano or an inorganic substituent; or R⁵ is C₁₋₆ alkyl, C₂₋₆ alkenyl, C₂₋₆ alkynyl, —CONHR¹, each optionally substituted by halo, carbonyl or one or more non-adjacent heteroatoms; or two adjacent R⁵ are linked to obtain a 5-6 membered optionally substituted carbocyclic or heterocyclic ring that may be fused to an additional optionally substituted carbocyclic or heterocyclic ring; and

n is 1-6.

In the above formula (TA4-1), B may be absent when Z¹ is N, or is H or a halogen when Z¹ is C.

In the above formula (TA4-1), W together with N and Z forms an optionally substituted 5- or 6-membered ring that is fused to an optionally substituted aryl or heteroaryl selected from the group consisting of:

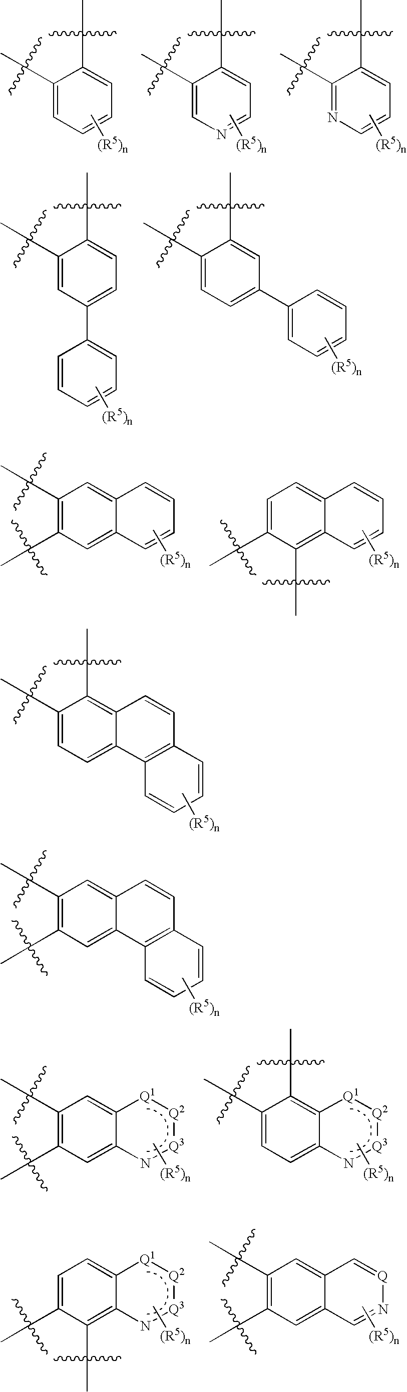

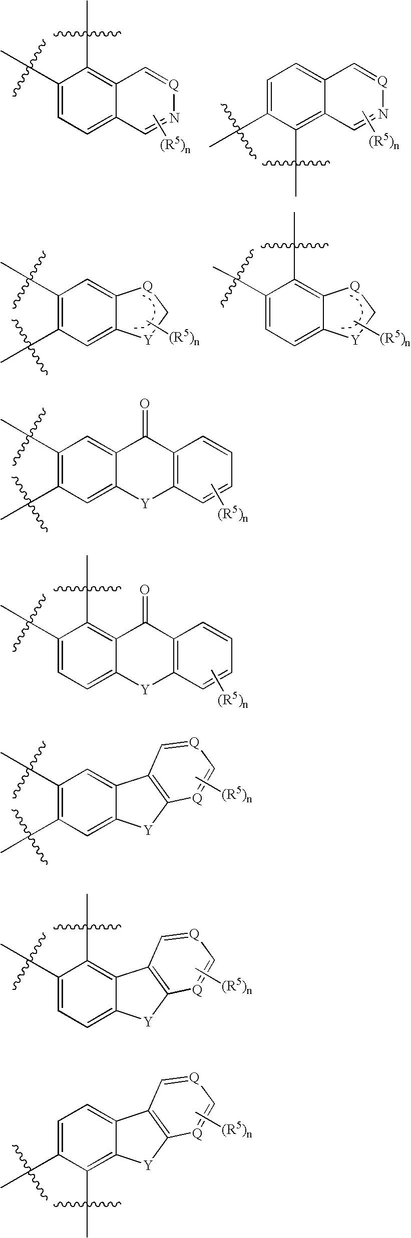

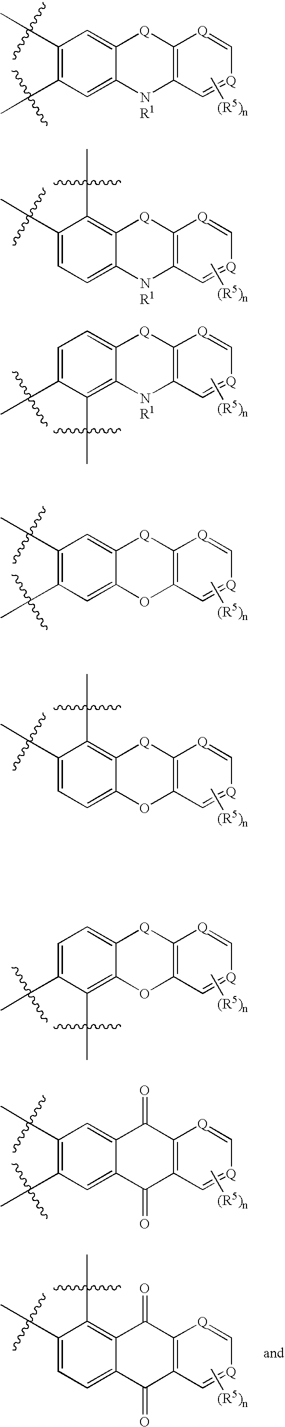

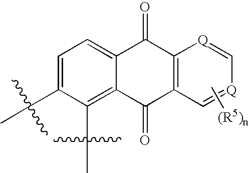

wherein each Q, Q¹, Q², and Q³ is independently CH or N;

Y is independently O, CH, C═O or NR¹;

n and R⁵ is as defined above.

In other embodiments, W together with N and Z form a group having the formula selected from the group consisting of

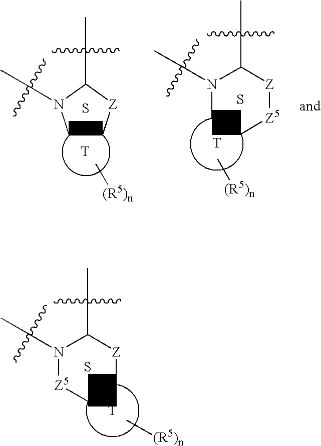

wherein Z is O, S, CR¹, NR¹, or C═O;
each Z⁵ is C(R⁶)₂, NR¹, or C═O, or Z and Z⁵ if adjacent can be —CR⁶═CR⁶— or —CR⁶═N—, and provided Z and Z⁵ if adjacent are not both NR¹;
each R¹ is H, C₁₋₆ alkyl, COR² or S(O)ₚR² wherein p is 1-2;
R is H, or a substituent known in the art, including but not limited to hydroxyl, alkyl, alkoxy, halo, amino, or amido; and
ring S and ring T may be saturated or unsaturated.

In some embodiments, W together with N and Z forms a 5- or 6-membered ring that is fused to a phenyl. In other embodiments, W together with N and Z forms a 5- or 6-membered ring that is optionally fused to another ring, when U is NR¹R², provided U is not NH₂. In certain embodiments, W together with N and Z forms a 5- or 6-membered ring that is not fused to another ring, when U is NR¹R² (e.g., NH₂).

In yet another embodiment, the compounds of the present invention have the general formula (TA4-2A) or (TA4-2B):

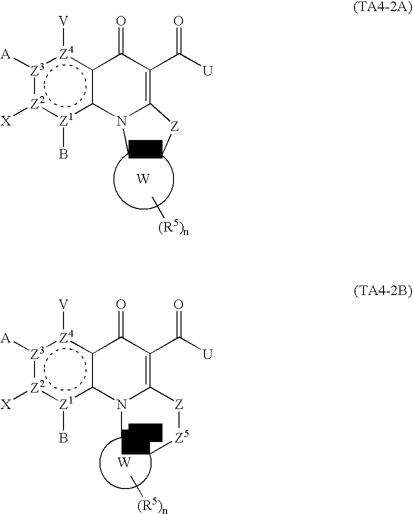

and pharmaceutically acceptable salts, esters and prodrugs thereof;

wherein A, B, V, X, U, Z, Z¹, Z², Z³, Z⁴ and n are as described for TA4-1;

Z⁵ is O, NR¹, CR⁶, or C═O;

R⁶ is H, C₁₋₆ alkyl, hydroxyl, alkoxy, halo, amino or amido; and

Z and Z⁵ may optionally form a double bond.

In the above formula (TA4-1), (TA4-2A) and (TA4-2B), U may be NR¹R², wherein R¹ is H, and R² is a C₁₋₁₀ alkyl optionally substituted with a heteroatom, a C₃₋₆ cycloalkyl, aryl or a 5-14 membered heterocyclic ring containing one or more N, O or S as a ring member. For example, R² may be a C₁₋₁₀ alkyl substituted with an optionally substituted morpholine, thiomorpholine, imidazole, aminodithiadazole, pyrrolidine, piperazine, pyridine or piperidine. In other examples, R¹ and R² together with N form an optionally substituted piperidine, pyrrolidine, piperazine, morpholine, thiomorpholine, imidazole, or aminodithiazole.

The compounds of formula (TA4-1), and methods of making and using them, are described in U.S. patent application Ser. No. 11/228,636, naming Whitten, et al., entitled QUINOLONE ANALOGS, and filed on Sep. 16, 2005. An example of a compound that significantly permeates the blood-brain barrier has a structure of the following general formula (TA4-1A):

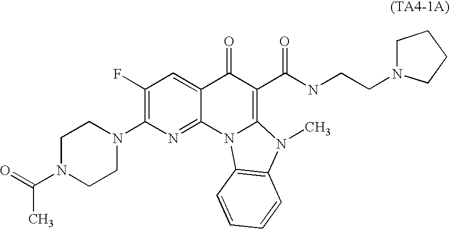

In yet another aspect, the therapeutic agent can be selected from compounds having the formula:

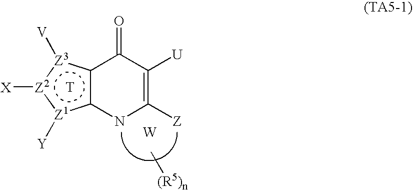

and pharmaceutically acceptable salts, esters and prodrugs thereof;

wherein V, X, and Y are absent if attached to a heteroatom other than Nitrogen, and independently H, halo, azido, R², CH₂R², SR², OR² or NR¹R² when attached to C or N; or

wherein V and X, or X and Y may form a carbocyclic ring, heterocyclic ring, aryl or heteroaryl, each of which may be optionally substituted and/or fused with a cyclic ring;

Z¹, Z² and Z³ are C, N, O or S, wherein among Z¹, Z² and Z³ there is at most one O atom, among Z¹, Z² and Z³ there is at most one S atom, and among Z¹, Z² and Z³ there is at most two carbon atoms;

Z is O, S, NR², CH₂ or C═O;

W together with N and Z forms an optionally substituted 5- or 6-membered ring that is fused to an optionally substituted aryl or heteroaryl, wherein said aryl or heteroaryl may be monocyclic or fused with a single or multiple ring, and wherein said ring optionally contains a heteroatom;

U is —C(═O)R², —COOR², —CONR¹R², —CONR¹—(CR¹₂)ₙ—NR³R⁴, SO₃R², SO₂NR¹R², SO₂NR¹NR¹R², SO₂NR¹OR², SO₂NR¹—(CR¹₂)ₙ—NR³R⁴ or SO₂NR¹NR¹—(CR¹₂)ₙ—NR³R⁴ or SO₂NR¹—O—(CR¹₂)ₙ—NR³R;

wherein in each NR¹R², R¹ and R² together with N may form an optionally substituted ring;

in NR³R⁴, R³ and R⁴ together with N may form an optionally substituted ring;

R¹ and R³ are independently H or C₁₋₆ alkyl;

each R² is H, or a C₁₋₁₀ alkyl or C₂₋₁₀ alkenyl each optionally substituted with a halogen, one or more non-adjacent heteroatoms selected from N, O and S, a carbocyclic ring, a heterocyclic ring, an aryl or heteroaryl, wherein each ring is optionally substituted; or R² is an optionally substituted carbocyclic ring, heterocyclic ring, aryl or heteroaryl; or R² is COR¹ or S(O)ₓR¹ wherein x is 1-2;

R⁴ is H, a C₁₋₁₀ alkyl or C₂₋₁₀ alkenyl optionally containing one or more non-adjacent heteroatoms selected from N, O and S, and optionally substituted with a carbocyclic or heterocyclic ring; or R³ and R⁴ together with N may form an optionally substituted ring;

each R⁵ is a substituent at any position on W; and is H, OR², amino, alkoxy, amido, halogen, cyano or an inorganic substituent; or R⁵ is C₁₋₆ alkyl, C₂₋₆ alkenyl, —CONHR¹, each optionally substituted by halo, carbonyl or one or more non-adjacent heteroatoms; or two adjacent R⁵ are linked to obtain a 5-6 membered optionally substituted carbocyclic or heterocyclic ring, optionally fused to an additional optionally substituted carbocyclic or heterocyclic ring; and

n is 1-6.

In the above formula (TA5-1), the ring labeled “T” is a five membered ring that can contain up to three heteroatoms selected from N, O, and S. Substituents V, X, and Y are as defined above, and each of them may be absent when the ring atom to which it is connected has no available open valence for substitution. The dashed circle indicates that each ring atom of ring T has a pi bond, which may be provided by either a heteroatom or an sp² hybridized carbon. In many embodiments, T is an aromatic ring, and in certain embodiments, T can be a non-aromatic ring. Ring “T” may, in some embodiments, form an optionally substituted 5-membered ring selected from the group consisting of:

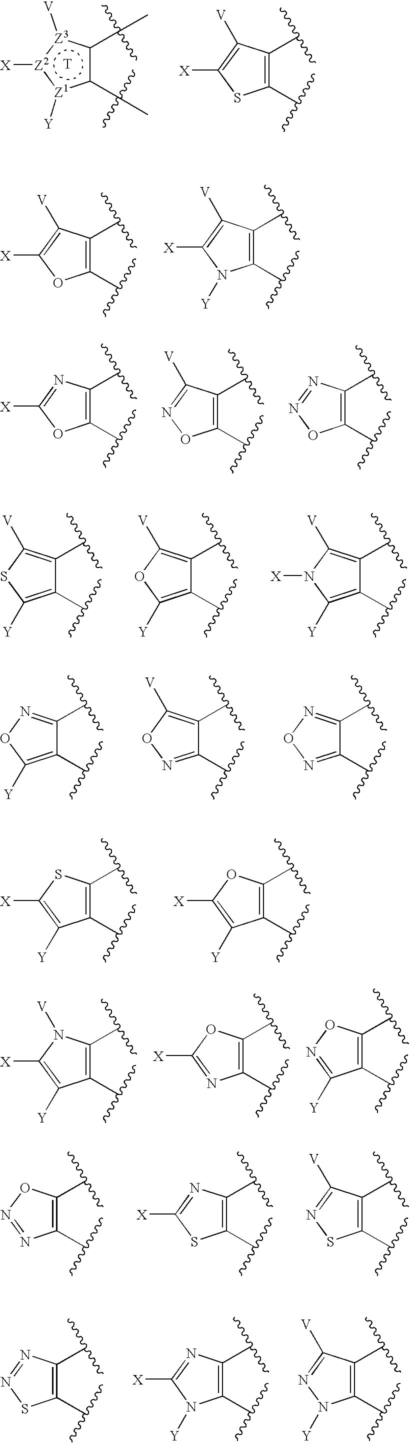

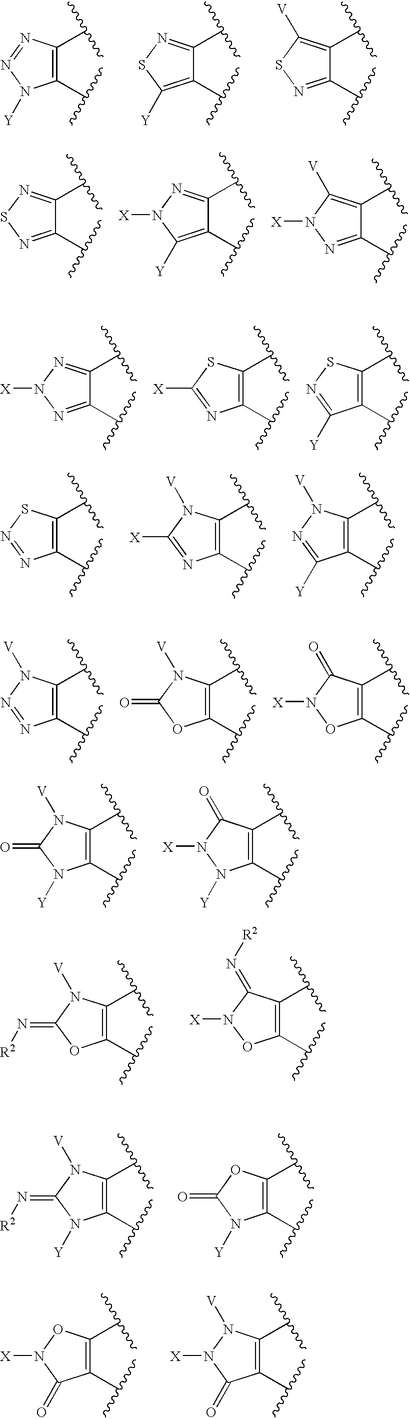

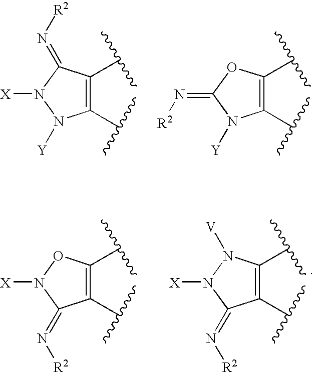

In the above formula (TA5-1), W together with N and Z may form an optionally substituted 5- or 6-membered aryl or heteroaryl ring that is fused to an optionally substituted aryl or heteroaryl selected from the group consisting of:

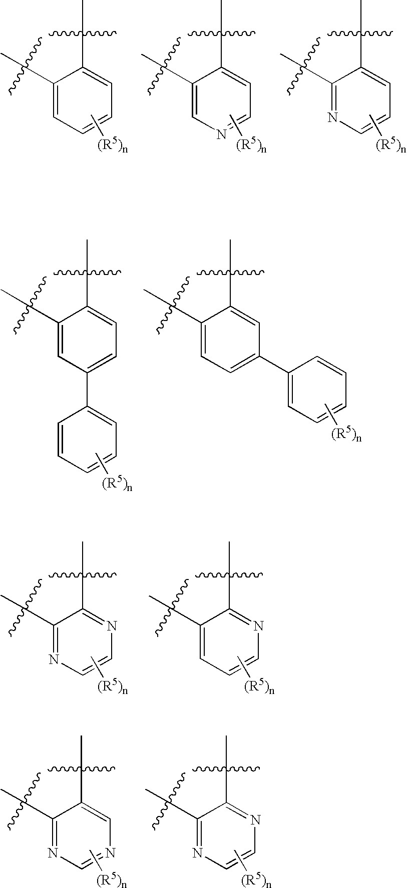

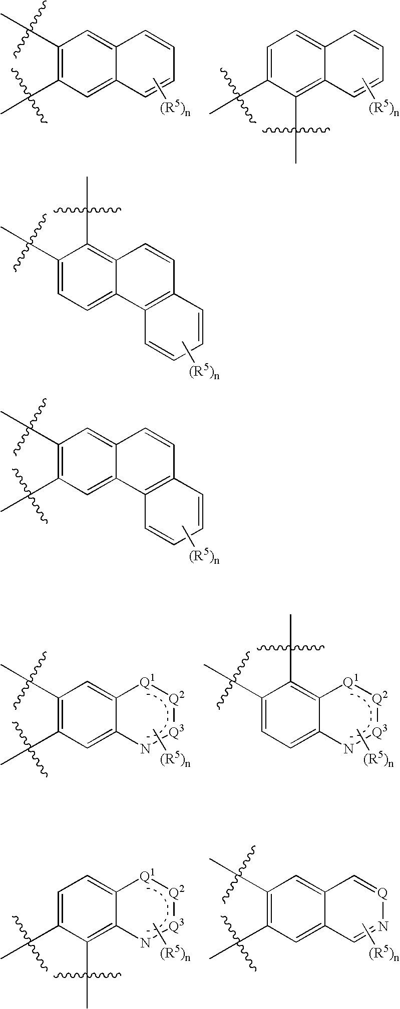

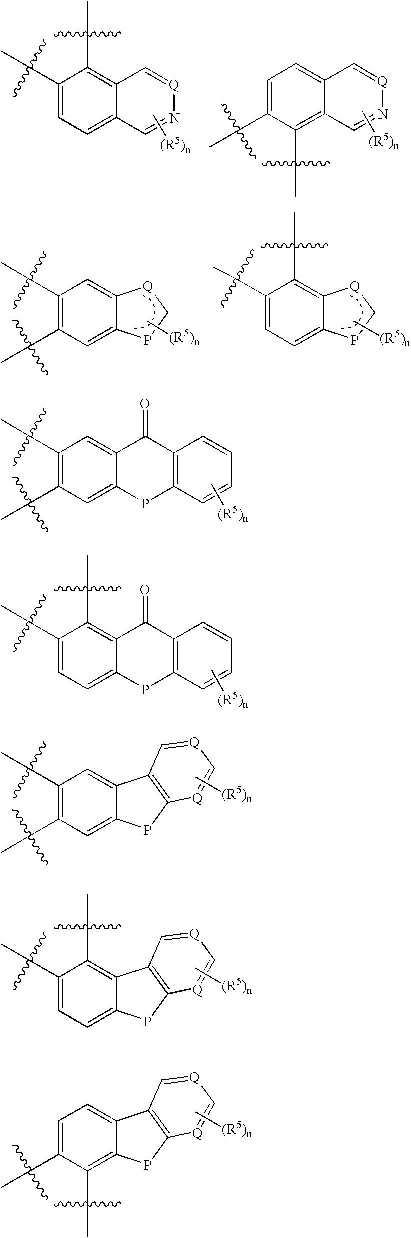

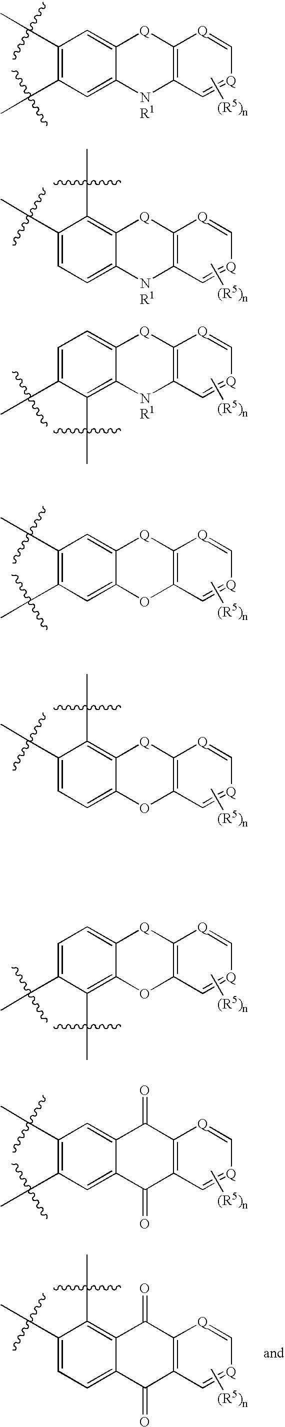

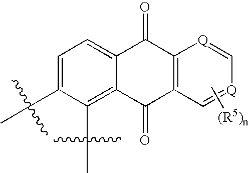

wherein each Q, Q¹, Q², and Q³ is independently CH or N;

P is independently O, CH, C═O or NR¹;

n and R⁵ are as defined above.

In other embodiments of these compounds, W together with N and Z may form a group having the formula selected from the group consisting of

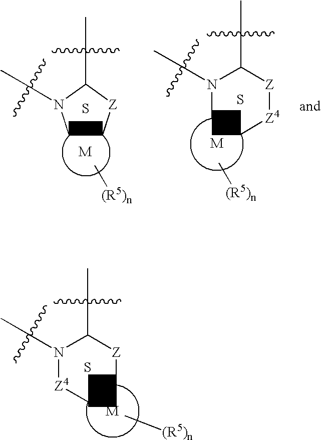

wherein Z is O, S, NR², CH₂ or C═O;

each Z⁴ is C(R⁶)₂, NR¹, or C═O, or Z and Z⁴ if adjacent can be —CR⁶═CR⁶— or —CR⁶═N—, and provided Z and Z⁴ if adjacent are not both NR¹;

R⁶ is H, or a substituent known in the art, including but not limited to hydroxyl, alkyl, alkoxy, halo, amino, or amido; and

Ring S and M may be saturated or unsaturated.

In some embodiments, W together with N and Z may form a 5- or 6-membered ring that is fused to a phenyl.

In yet another embodiment, the compounds of the present invention have the general formula (TA5-2A) or (TA5-2B):

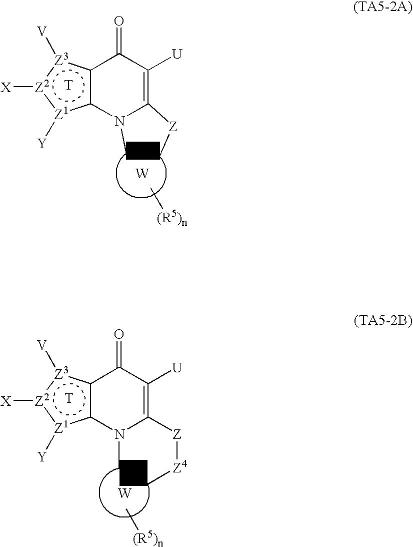

and pharmaceutically acceptable salts, esters and prodrugs thereof;

wherein U, V, W, X, Y, Z, Z¹, Z², Z³, R⁵ and n are as described above for TA5-1;

Z⁴ is CR⁶, NR², or C═O; and

Z and Z⁴ may optionally form a double bond.

In the above formula (TA5-1), (TA5-2A) and (TA5-2B), U may be SO₂NR¹R², wherein R¹ is H, and R² is a C₁₋₁₀ alkyl optionally substituted with a heteroatom, a C₃₋₆ cycloalkyl, aryl or a 5-14 membered heterocyclic ring containing one or more N, O or S. For example, R² may be a C₁₋₁₀ alkyl substituted with an optionally substituted morpholine, thiomorpholine, imidazole, aminodithiadazole, pyrrolidine, piperazine, pyridine or piperidine. In other examples, R¹ and R² together with N form an optionally substituted piperidine, pyrrolidine, piperazine, morpholine, thiomorpholine, imidazole, or aminodithiazole.

In other embodiments of these compounds, U is SO₂NR¹—(CR¹₂)ₙ—NR³R⁴; n is 1-4; each R¹ is H or alkyl; and R³ and R⁴ in NR³R⁴ together form an optionally substituted piperidine, pyrrolidine, piperazine, morpholine, thiomorpholine, imidazole, or aminodithiazole. In some examples, U is SO₂NH—(CH₂)ₙ—NR³R⁴ wherein R³ and R⁴ together with N form an optionally substituted pyrrolidine, which may be linked to (CH₂)ₙ at any position in the pyrrolidine ring. In one embodiment, R³ and R⁴ together with N form an N-methyl substituted pyrrolidine.

In one embodiment, the present invention provides compounds having formula (TA5-1), (TA5-2A) or (TA5-2B), wherein:

each of V and Y if present is independently H or halogen (e.g., chloro or fluoro);

X is —(R⁵)R¹R², wherein R⁵ is C or N and wherein in each —(R⁵)R¹R² , R¹ and R² together may form an optionally substituted aryl or heteroaryl ring;

Z is NH or N-alkyl (e.g., N—CH₃);

W together with N and Z forms an optionally substituted 5- or 6-membered ring that is fused with an optionally substituted aryl or heteroaryl ring; and

U is —SO₂R⁵R⁶—(CH₂)ₙ—CHR²—NR³R⁴, wherein R⁵ is CR¹ or N; R¹ is H or alkyl; R⁶ is H or C₁₋₁₀ alkyl and wherein in the —CHR²—NR³R⁴ moiety each R³ or R⁴ together with the C may form an optionally substituted heterocyclic or heteroaryl ring, or wherein in the —CHR²—NR³R⁴ moiety each R³ or R⁴ together with the N may form an optionally substituted carbocyclic, heterocyclic, aryl or heteroaryl ring.

In another embodiment, the present invention provides compounds having formula (TA5-1), (TA5-2A) or (TA5-2B), wherein:

V and Y if present is H or halogen (e.g., chloro or fluoro);

X if present is —(CR¹)R¹R² or NR¹R², wherein R¹ and R² together may form an optionally substituted aryl or heteroaryl ring;

Z is NH or N-alkyl (e.g., N—CH₃);

W together with N and Z forms an optionally substituted 5- or 6-membered ring that is fused with an optionally substituted aryl or heteroaryl ring; and

U is —SO₂NR⁶—(CH₂)ₙ—CHR²—NR³R⁴ or —SO₂CR¹R⁶—(CH₂)ₙ—CHR²—NR³R⁴;

R⁶ is H or alkyl and wherein in the —CHR²—NR³R⁴ moiety each R³ or R⁴ together with the C may form an optionally substituted heterocyclic or heteroaryl ring, or wherein in the —CHR²—NR³R⁴ moiety each R³ or R⁴ together with the N may form an optionally substituted carbocyclic, heterocyclic, aryl or heteroaryl ring.

In yet another embodiment, the compounds of the present invention have the general formula (TA5-3):

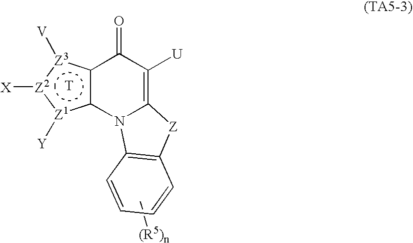

and pharmaceutically acceptable salts, esters and prodrugs thereof;

wherein U, V, X, Y, Z, Z¹, Z², Z³, R⁵ and n are as described above.

In yet another embodiment, the compounds of the present invention have the general formula (TA5-4A) or (TA5-4B):

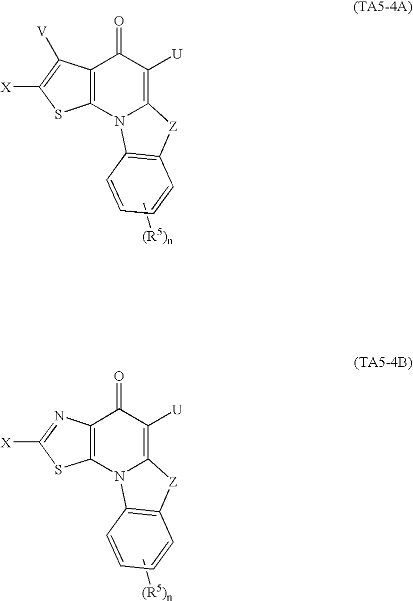

and pharmaceutically acceptable salts, esters and prodrugs thereof;

wherein U, V, X, Z, R⁵ and n are as described above for TA5- 1.

Compounds of Formula (TA5-1), and methods for making and using them, are described in International Patent Application No. PCT/US07/70774, filed Jun. 8, 2007, naming Pierre, et al., and entitled PYRIDINONE ANALOGS.

In still another aspect, the therapeutic agent for the combinations of the invention can be a compound of the formula:

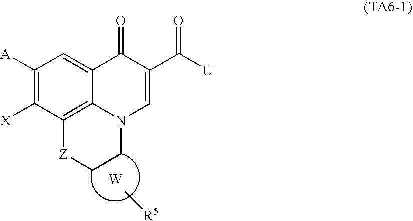

and pharmaceutically acceptable salts, esters and prodrugs thereof,

wherein X is H, OR², NR¹R², halogen, azido, SR² or CH₂R;

A is H, halogen, NR¹R², SR², OR², CH₂R², azido or NR¹—(CR¹₂)ₙ—NR³R⁴;

Z is O, S, NR¹ or CH₂;

U is R², OR², NR¹R² or NR¹—(CR¹₂)ₙ—NR³R⁴ provided U is not H;

W is an optionally substituted aryl or heteroaryl, which may be monocyclic or fused with a single or multiple ring optionally containing a heteroatom;

wherein R¹ and R² together with N in NR¹R², and R³and R⁴together with N in NR³R⁴ may independently form an optionally substituted 5-6 membered ring containing N, and optionally O or S;

R¹ and R³ are independently H or a C₁₋₆ alkyl; and

R² and R⁴ are independently H, or a C₁₋₁₀ alkyl or C₂₋₁₀ alkenyl optionally containing one or more non-adjacent heteroatoms selected from N, O, and S, and optionally substituted with a substituted or unsubstituted aryl, heteroaryl, carbocyclic, or heterocyclic ring; or R² is an optionally cycloalkyl, substituted heterocyclic ring, aryl or heteroaryl;

R⁵ is a substituent at any position of W and is H, halo, cyano, azido, —CONHR¹, OR², or C₁₋₆ alkyl or C₂₋₆ alkenyl, each optionally substituted by halo, ═O or one or more heteroatoms;

provided X and A both are not H, and further provided that R⁵ is cyano or —CONHR¹ when A is H, halogen or NR¹R²;

or a compound having formula (TA6-1A)

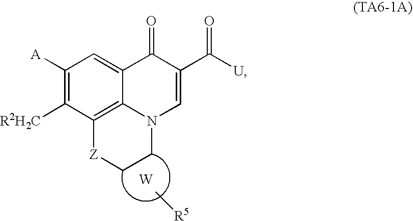

and pharmaceutically acceptable salts, esters and prodrugs thereof;

A is H, halogen, azido, SR², OR², CH₂R², NR¹R², or NR¹—(CR¹₂)ₙ—NR³R⁴;

Z, U, W, R¹, R², R³ and R⁴ are as defined in formula TA6-1; and

R⁵ is a substituent at any position of W and is H, halo, cyano, azido, —CONHR¹, OR², or C₁₋₆ alkyl or C₂₋₆ alkenyl, each optionally substituted by halo, ═O or one or more heteroatoms;

wherein each optionally substituted moiety in formula TA6-1 and -1A is substituted with one or more halo, cyano, azido, acetyl, amido, OR², NR¹R², carbamate, C₁₋₁₀ alkyl, C₂₋₁₀ alkenyl, each optionally substituted by halo, ═O, aryl or one or more heteroatoms selected from N, O and S; or is substituted with an aryl, a carbocyclic or a heterocyclic ring.

In the above formula TA6-1 or TA6-1A, W may be selected from the group consisting of

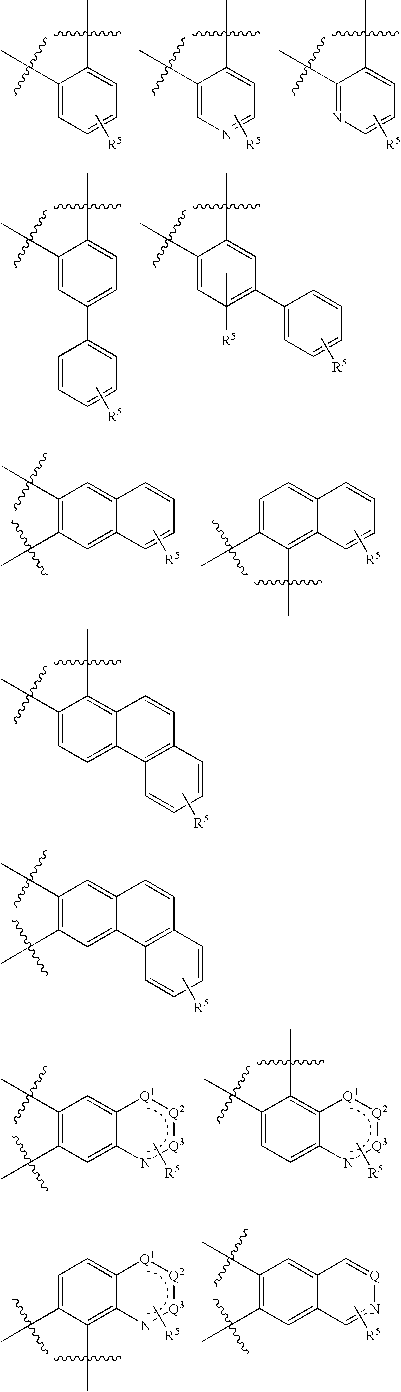

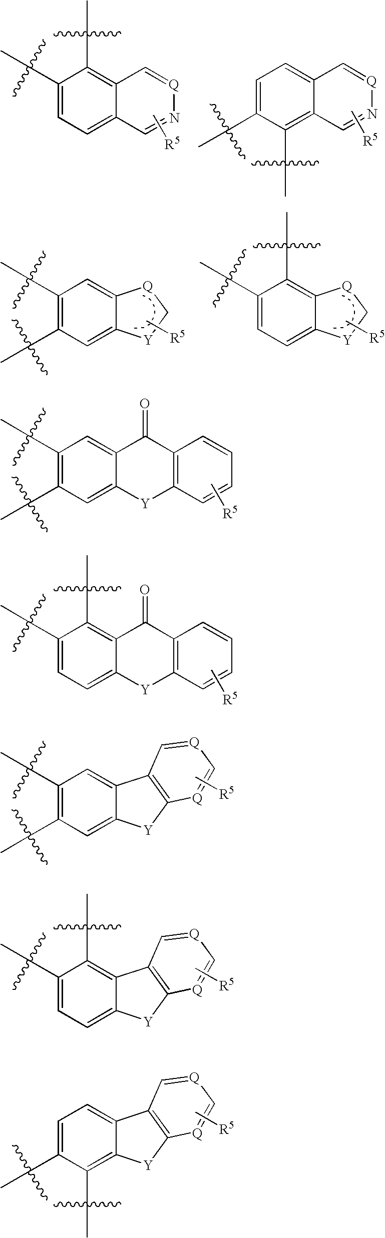

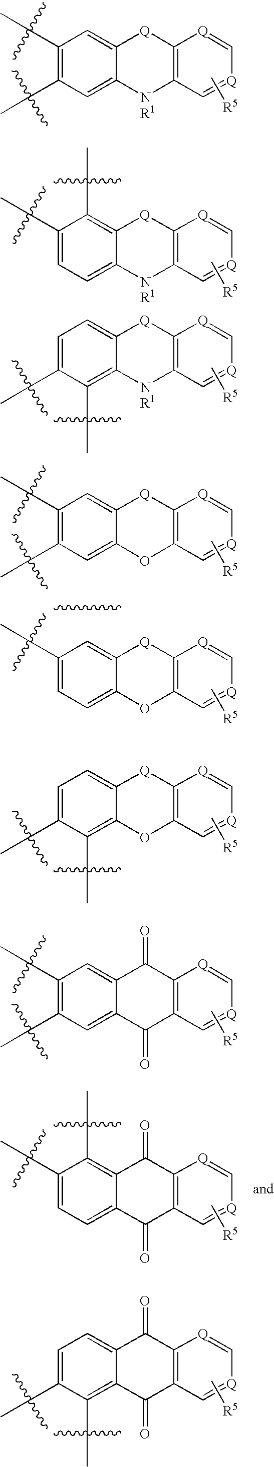

wherein Q, Q¹, Q², and Q³ are independently CH or N;

Y is independently O, CH, ═O or NR¹; and

R⁵ is as defined in formula 1.

In some embodiments of these compounds, each Win the above formula TA6-1 or TA6-1A may be an optionally substituted phenyl, pyridine, biphenyl, naphthalene, phenanthrene, quinoline, isoquinoline, quinazoline, cinnoline, phthalazine, quinoxaline, indole, benzimidazole, benzoxazole, benzthiazole, benzofuran, anthrone, xanthone, acridone, fluorenone, carbazolyl, pyrimido[4,3-b]furan, pyrido[4,3-b]indole, pyrido[2,3-b]indole, dibenzofuran, acridine or acridizine. In one embodiment, W is an optionally substituted phenyl.

The compounds of formula (TA6-1), and methods for making and using them, are described in U.S. patent application Ser. No. 11/404,947, to Whitten, et al., which was filed on Apr. 14, 2006, and is entitled QUINOBENZOXAZINE ANALOGS AND METHODS OF USING THEREOF.

A compound of general formula TA1-1A or TA1-1B is a preferred therapeutic agent for use in the methods and compositions of the invention. More detail on methods for the formulation and administration of such compounds are provided in U.S. Provisional application Ser. No. 11/757,273, filed Jun. 1, 2007, naming Lim et al., and entitled DRUG ADMINISTRATION METHODS.

“Optionally substituted” as used herein indicates that the particular group or groups being described may have no non-hydrogen substituents, or the group or groups may have one or more non-hydrogen substituents. If not otherwise specified, the total number of such substituents that may be present is equal to the number of H atoms present on the unsubstituted form of the group being described. Where an optional substituent is attached via a double bond, such as a carbonyl oxygen (═O), the group takes up two available valences, so the total number of substituents that may be included is reduced according to the number of available valences.

The compounds of the invention often have ionizable groups so as to be capable of preparation as salts. In that case, wherever reference is made to the compound, it is understood in the art that a pharmaceutically acceptable salt may also be used. These salts may be acid addition salts involving inorganic or organic acids or the salts may, in the case of acidic forms of the compounds of the invention be prepared from inorganic or organic bases. Frequently, the compounds are prepared or used as pharmaceutically acceptable salts prepared as addition products of pharmaceutically acceptable acids or bases. Suitable pharmaceutically acceptable acids and bases are well-known in the art, such as hydrochloric, sulphuric, hydrobromic, acetic, lactic, citric, or tartaric acids for forming acid addition salts, and potassium hydroxide, sodium hydroxide, ammonium hydroxide, caffeine, various amines, and the like for forming basic salts. Methods for preparation of the appropriate salts are well-established in the art. In some cases, the compounds may contain both an acidic and a basic functional group, in which case they may have two ionized groups and yet have no net charge.

In some cases, the compounds of the invention contain one or more chiral centers. The invention includes each of the isolated stereoisomeric forms as well as mixtures of stereoisomers in varying degrees of chiral purity, including racemic mixtures. It also encompasses the various diastereomers and tautomers that can be formed. The compounds of the invention may also exist in more than one tautomeric form; the depiction herein of one tautomer is for convenience only, and is also understood to encompass other tautomers of the form shown.

As used herein, the terms “alkyl,” “alkenyl” and “alkynyl” include straight-chain, branched-chain and cyclic monovalent hydrocarbyl radicals, and combinations of these, which contain only C and H when they are unsubstituted. Examples include methyl, ethyl, isobutyl, cyclohexyl, cyclopentylethyl, 2-propenyl, 3-butynyl, and the like. The total number of carbon atoms in each such group is sometimes described herein, e.g., when the group can contain up to ten carbon atoms it can be represented as 1-10C or as C1-C10 or C1-10. When heteroatoms (N, O and S typically) are allowed to replace carbon atoms as in heteroalkyl groups, for example, the numbers describing the group, though still written as e.g. C1-C6, represent the sum of the number of carbon atoms in the group plus the number of such heteroatoms that are included as replacements for carbon atoms in the backbone of the ring or chain being described.

Typically, the alkyl, alkenyl and alkynyl substituents of the invention contain 1-10C (alkyl) or 2-10C (alkenyl or alkynyl). Preferably they contain 1-8C (alkyl) or 2-8C (alkenyl or alkynyl). Sometimes they contain 1-4C (alkyl) or 2-4C (alkenyl or alkynyl). A single group can include more than one type of multiple bond, or more than one multiple bond; such groups are included within the definition of the term “alkenyl” when they contain at least one carbon-carbon double bond, and are included within the term “alkynyl” when they contain at least one carbon-carbon triple bond.

Alkyl, alkenyl and alkynyl groups are often optionally substituted to the extent that such substitution makes sense chemically. Typical substituents include, but are not limited to, halo, ═O, ═N—CN, ═N—OR, ═NR, OR, NR₂, SR, SO₂R, SO₂NR₂, NRSO₂R, NRCONR₂, NRCOOR, NRCOR, CN, COOR, CONR₂, OOCR, COR, and NO₂, wherein each R is independently H, C1-C8 alkyl, C2-C8 heteroalkyl, C1-C8 acyl, C2-C8 heteroacyl, C2-C8 alkenyl, C2-C8 heteroalkenyl, C2-C8 alkynyl, C2-C8 heteroalkynyl, C6-C10 aryl, or C5-C10 heteroaryl, and each R is optionally substituted with halo, ═O, ═N—CN, ═N—OR′, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂, wherein each R′ is independently H, C1-C8 alkyl, C2-C8 heteroalkyl, C1-C8 acyl, C2-C8 heteroacyl, C6-C10 aryl or C5-C10 heteroaryl. Alkyl, alkenyl and alkynyl groups can also be substituted by C1-C8 acyl, C2-C8 heteroacyl, C6-C10 aryl or C5-C10 heteroaryl, each of which can be substituted by the substituents that are appropriate for the particular group.

“Acetylene” substituents are 2-10C alkynyl groups that are optionally substituted, and are of the formula —C≡C—R^{a}, wherein R^{a} is H or C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, or C6-C12 heteroarylalkyl,

and each R^{a} group is optionally substituted with one or more substituents selected from halo, ═O, ═N—CN, ═N—OR′, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂,

wherein each R′ is independently H, C1-C6 alkyl, C2-C6 heteroalkyl, C1-C6 acyl, C2-C6 heteroacyl, C6-C10 aryl, C5-C10 heteroaryl, C7-12 arylalkyl, or C6-12 heteroarylalkyl, each of which is optionally substituted with one or more groups selected from halo, C1-C4 alkyl, C1-C4 heteroalkyl, C1-C6 acyl, C1-C6 heteroacyl, hydroxy, amino, and ═O; and

wherein two R′ can be linked to form a 3-7 membered ring optionally containing up to three heteroatoms selected from N, O and S. In some embodiments, R^{a} of —C≡C—R^{a} is H or Me.

“Heteroalkyl”, “heteroalkenyl”, and “heteroalkynyl” and the like are defined similarly to the corresponding hydrocarbyl (alkyl, alkenyl and alkynyl) groups, but the ‘hetero’ terms refer to groups that contain 1-3 O, S or N heteroatoms or combinations thereof within the backbone residue; thus at least one carbon atom of a corresponding alkyl, alkenyl, or alkynyl group is replaced by one of the specified heteroatoms to form a heteroalkyl, heteroalkenyl, or heteroalkynyl group. The typical and preferred sizes for heteroforms of alkyl, alkenyl and alkynyl groups are generally the same as for the corresponding hydrocarbyl groups, and the substituents that may be present on the heteroforms are the same as those described above for the hydrocarbyl groups. For reasons of chemical stability, it is also understood that, unless otherwise specified, such groups do not include more than two contiguous heteroatoms except where an oxo group is present on N or S as in a nitro or sulfonyl group.

While “alkyl” as used herein includes cycloalkyl and cycloalkylalkyl groups, the term “cycloalkyl” may be used herein to describe a carbocyclic non-aromatic group that is connected via a ring carbon atom, and “cycloalkylalkyl” may be used to describe a carbocyclic non-aromatic group that is connected to the molecule through an alkyl linker. Similarly, “heterocyclyl” may be used to describe a non-aromatic cyclic group that contains at least one heteroatom as a ring member and that is connected to the molecule via a ring atom, which may be C or N; and “heterocyclylalkyl” may be used to describe such a group that is connected to another molecule through a linker. The sizes and substituents that are suitable for the cycloalkyl, cycloalkylalkyl, heterocyclyl, and heterocyclylalkyl groups are the same as those described above for alkyl groups. As used herein, these terms also include rings that contain a double bond or two, as long as the ring is not aromatic.

As used herein, “acyl” encompasses groups comprising an alkyl, alkenyl, alkynyl, aryl or arylalkyl radical attached at one of the two available valence positions of a carbonyl carbon atom, and heteroacyl refers to the corresponding groups wherein at least one carbon other than the carbonyl carbon has been replaced by a heteroatom chosen from N, O and S. Thus heteroacyl includes, for example, —C(═O)OR and —C(═O)NR₂ as well as —C(═O)-heteroaryl.

Acyl and heteroacyl groups are bonded to any group or molecule to which they are attached through the open valence of the carbonyl carbon atom. Typically, they are C1-C8 acyl groups, which include formyl, acetyl, pivaloyl, and benzoyl, and C2-C8 heteroacyl groups, which include methoxyacetyl, ethoxycarbonyl, and 4-pyridinoyl. The hydrocarbyl groups, aryl groups, and heteroforms of such groups that comprise an acyl or heteroacyl group can be substituted with the substituents described herein as generally suitable substituents for each of the corresponding component of the acyl or heteroacyl group.

“Aromatic” moiety or “aryl” moiety refers to a monocyclic or fused bicyclic moiety having the well-known characteristics of aromaticity; examples include phenyl and naphthyl. Similarly, “heteroaromatic” and “heteroaryl” refer to such monocyclic or fused bicyclic ring systems which contain as ring members one or more heteroatoms selected from O, S and N. The inclusion of a heteroatom permits aromaticity in 5-membered rings as well as 6-membered rings. Typical heteroaromatic systems include monocyclic C5-C6 aromatic groups such as pyridyl, pyrimidyl, pyrazinyl, thienyl, furanyl, pyrrolyl, pyrazolyl, thiazolyl, oxazolyl, and imidazolyl and the fused bicyclic moieties formed by fusing one of these monocyclic groups with a phenyl ring or with any of the heteroaromatic monocyclic groups to form a C8-C10 bicyclic group such as indolyl, benzimidazolyl, indazolyl, benzotriazolyl, isoquinolyl, quinolyl, benzothiazolyl, benzofuranyl, pyrazolopyridyl, quinazolinyl, quinoxalinyl, cinnolinyl, and the like. Any monocyclic or fused ring bicyclic system which has the characteristics of aromaticity in terms of electron distribution throughout the ring system is included in this definition. It also includes bicyclic groups where at least the ring which is directly attached to the remainder of the molecule has the characteristics of aromaticity. Typically, the ring systems contain 5-12 ring member atoms. Preferably the monocyclic heteroaryls contain 5-6 ring members, and the bicyclic heteroaryls contain 8-10 ring members.

Aryl and heteroaryl moieties may be substituted with a variety of substituents including C1-C8 alkyl, C2-C8 alkenyl, C2-C8 alkynyl, C5-C12 aryl, C1-C8 acyl, and heteroforms of these, each of which can itself be further substituted; other substituents for aryl and heteroaryl moieties include halo, OR, NR₂, SR, SO₂R, SO₂NR₂, NRSO₂R, NRCONR₂, NRCOOR, NRCOR, CN, COOR, CONR₂, OOCR, COR, and NO₂, wherein each R is independently H, C1-C8 alkyl, C2-C8 heteroalkyl, C2-C8 alkenyl, C2-C8 heteroalkenyl, C2-C8 alkynyl, C2-C8 heteroalkynyl, C6-C10 aryl, C5-C10 heteroaryl, C7-C12 arylalkyl, or C6-C12 heteroarylalkyl, and each R is optionally substituted as described above for alkyl groups. The substituent groups on an aryl or heteroaryl group may of course be further substituted with the groups described herein as suitable for each type of such substituents or for each component of the substituent. Thus, for example, an arylalkyl substituent may be substituted on the aryl portion with substituents described herein as typical for aryl groups, and it may be further substituted on the alkyl portion with substituents described herein as typical or suitable for alkyl groups.

Similarly, “arylalkyl” and “heteroarylalkyl” refer to aromatic and heteroaromatic ring systems which are bonded to their attachment point through a linking group such as an alkylene, including substituted or unsubstituted, saturated or unsaturated, cyclic or acyclic linkers. Typically the linker is C1-C8 alkyl or a hetero form thereof. These linkers may also include a carbonyl group, thus making them able to provide substituents as an acyl or heteroacyl moiety. An aryl or heteroaryl ring in an arylalkyl or heteroarylalkyl group may be substituted with the same substituents described above for aryl groups. Preferably, an arylalkyl group includes a phenyl ring optionally substituted with the groups defined above for aryl groups and a C1-C4 alkylene that is unsubstituted or is substituted with one or two C1-C4 alkyl groups or heteroalkyl groups, where the alkyl or heteroalkyl groups can optionally cyclize to form a ring such as cyclopropane, dioxolane, or oxacyclopentane. Similarly, a heteroarylalkyl group preferably includes a C5-C6 monocyclic heteroaryl group that is optionally substituted with the groups described above as substituents typical on aryl groups and a C1-C4 alkylene that is unsubstituted or is substituted with one or two C1-C4 alkyl groups or heteroalkyl groups, or it includes an optionally substituted phenyl ring or C5-C6 monocyclic heteroaryl and a C1-C4 heteroalkylene that is unsubstituted or is substituted with one or two C1-C4 alkyl or heteroalkyl groups, where the alkyl or heteroalkyl groups can optionally cyclize to form a ring such as cyclopropane, dioxolane, or oxacyclopentane.

Where an arylalkyl or heteroarylalkyl group is described as optionally substituted, the substituents may be on either the alkyl or heteroalkyl portion or on the aryl or heteroaryl portion of the group. The substituents optionally present on the alkyl or heteroalkyl portion are the same as those described above for alkyl groups generally; the substituents optionally present on the aryl or heteroaryl portion are the same as those described above for aryl groups generally.

“Arylalkyl” groups as used herein are hydrocarbyl groups if they are unsubstituted, and are described by the total number of carbon atoms in the ring and alkylene or similar linker. Thus a benzyl group is a C7-arylalkyl group, and phenylethyl is a C8-arylalkyl.

“Heteroarylalkyl” as described above refers to a moiety comprising an aryl group that is attached through a linking group, and differs from “arylalkyl” in that at least one ring atom of the aryl moiety or one atom in the linking group is a heteroatom selected from N, O and S. The heteroarylalkyl groups are described herein according to the total number of atoms in the ring and linker combined, and they include aryl groups linked through a heteroalkyl linker; heteroaryl groups linked through a hydrocarbyl linker such as an alkylene; and heteroaryl groups linked through a heteroalkyl linker. Thus, for example, C7-heteroarylalkyl would include pyridylmethyl, phenoxy, and N-pyrrolylmethoxy.

“Alkylene” as used herein refers to a divalent hydrocarbyl group; because it is divalent, it can link two other groups together. Typically it refers to —(CH₂)ₙ— where n is 1-8 and preferably n is 1-4, though where specified, an alkylene can also be substituted by other groups, and can be of other lengths, and the open valences need not be at opposite ends of a chain. Thus —CH(Me)— and —C(Me)₂— may also be referred to as alkylenes, as can a cyclic group such as cyclopropan-1,1-diyl. Where an alkylene group is substituted, the substituents include those typically present on alkyl groups as described herein.

In general, any alkyl, alkenyl, alkynyl, acyl, or aryl or arylalkyl group or any heteroform of one of these groups that is contained in a substituent may itself optionally be substituted by additional substituents. The nature of these substituents is similar to those recited with regard to the primary substituents themselves if the substituents are not otherwise described. Thus, where an embodiment of, for example, R⁷ is alkyl, this alkyl may optionally be substituted by the remaining substituents listed as embodiments for R⁷ where this makes chemical sense, and where this does not undermine the size limit provided for the alkyl per se; e.g., alkyl substituted by alkyl or by alkenyl would simply extend the upper limit of carbon atoms for these embodiments, and is not included. However, alkyl substituted by aryl, amino, alkoxy, ═O, and the like would be included within the scope of the invention, and the atoms of these substituent groups are not counted in the number used to describe the alkyl, alkenyl, etc. group that is being described. Where no number of substituents is specified, each such alkyl, alkenyl, alkynyl, acyl, or aryl group may be substituted with a number of substituents according to its available valences; in particular, any of these groups may be substituted with fluorine atoms at any or all of its available valences, for example.

“Heteroform” as used herein refers to a derivative of a group such as an alkyl, aryl, or acyl, wherein at least one carbon atom of the designated carbocyclic group has been replaced by a heteroatom selected from N, O and S. Thus the heteroforms of alkyl, alkenyl, alkynyl, acyl, aryl, and arylalkyl are heteroalkyl, heteroalkenyl, heteroalkynyl, heteroacyl, heteroaryl, and heteroarylalkyl, respectively. It is understood that no more than two N, O or S atoms are ordinarily connected sequentially, except where an oxo group is attached to N or S to form a nitro or sulfonyl group.

“Halo”, as used herein includes fluoro, chloro, bromo and iodo. Fluoro and chloro are often preferred.

“Amino” as used herein refers to NH₂, but where an amino is described as “substituted” or “optionally substituted”, the term includes NR′R″ wherein each R′ and R″ is independently H, or is an alkyl, alkenyl, alkynyl, acyl, aryl, or arylalkyl group or a heteroform of one of these groups, and each of the alkyl, alkenyl, alkynyl, acyl, aryl, or arylalkyl groups or heteroforms of one of these groups is optionally substituted with the substituents described herein as suitable for the corresponding group. The term also includes forms wherein R′ and R″ are linked together to form a 3-8 membered ring which may be saturated, unsaturated or aromatic and which contains 1-3 heteroatoms independently selected from N, O and S as ring members, and which is optionally substituted with the substituents described as suitable for alkyl groups or, if NR′R″ is an aromatic group, it is optionally substituted with the substituents described as typical for heteroaryl groups.

As used herein, the term “carbocycle” refers to a cyclic compound containing only carbon atoms in the ring, whereas a “heterocycle” refers to a cyclic compound comprising a heteroatom. The carbocyclic and heterocyclic structures encompass compounds having monocyclic, bicyclic or multiple ring systems.

As used herein, the term “heteroatom” refers to any atom that is not carbon or hydrogen, such as nitrogen, oxygen or sulfur.

Illustrative examples of heterocycles include but are not limited to tetrahydrofuran, 1,3 dioxolane, 2,3 dihydrofuran, pyran, tetrahydropyran, benzofuran, isobenzofuran, 1,3 dihydro isobenzofuran, isoxazole, 4,5 dihydroisoxazole, piperidine, pyrrolidine, pyrrolidin 2 one, pyrrole, pyridine, pyrimidine, octahydro pyrrolo[3,4b]pyridine, piperazine, pyrazine, morpholine, thiomorpholine, imidazole, imidazolidine 2,4 dione, 1,3 dihydrobenzimidazol 2 one, indole, thiazole, benzothiazole, thiadiazole, thiophene, tetrahydro thiophene 1,1 dioxide, diazepine, triazole, guanidine, diazabicyclo[2.2.1]heptane,2,5 diazabicyclo[2.2.1]heptane,2,3,4,4a,9,9a hexahydro 1H β carboline, oxirane, oxetane, tetrahydropyran, dioxane, lactones, aziridine, azetidine, piperidine, lactams, and may also encompass heteroaryls. Other illustrative examples of heteroaryls include but are not limited to furan, pyrrole, pyridine, pyrimidine, imidazole, benzimidazole and triazole.

As used herein, the term “inorganic substituent” refers to substituents that do not contain carbon or contain carbon bound to elements other than hydrogen (e.g., elemental carbon, carbon monoxide, carbon dioxide, and carbonate). Examples of inorganic substituents include but are not limited to nitro, halogen, azido, cyano, sulfonyls, sulfinyls, sulfonates, phosphates, and the like.

Conditions Associated with Aberrant Cell Proliferation

The invention in part provides methods for inhibiting cell proliferation and methods for treating a condition related to aberrant cell proliferation. For example, provided are methods of treating a cell proliferative condition in a subject, which comprises administering a therapeutic agent described herein to a subject in need of a treatment for a cell proliferative disorder; whereby the therapeutic agent is administered in an amount effective to treat the cell proliferative condition. The subject may be a research animal (e.g., rodent, dog, cat, monkey), optionally containing a tumor such as a xenograft tumor (e.g., human tumor), for example, or may be a human.

Cell proliferative conditions include different types of cancers. Cancers are a leading cause of human death worldwide. In the United States alone, cancers cause the death of well over a half-million people annually, with some 1.4 million new cases diagnosed per year. Worldwide, several cancers stand out as the leading killers. In particular, carcinomas of the lung, prostate, breast, colon/rectum, pancreas, kidney, central nervous system (CNS) and ovary represent the causes of cancer death. A cell proliferative condition sometimes is a tumor or non-tumor cancer, including but not limited to, cancers of the colorectum, breast, ovary, lung, thymus, liver, pancreas, lymph node, stomach, appendix, small bowel (i.e., duodenum, jejunum, and ileum), colon, rectum, prostate, brain, head and neck, skin, kidney, heart, adrenal, pituitary, parathyroid, thyroid, bone marrow and blood (e.g., leukemia, lymphoma, carcinoma, multiple myeloma). Provided hereafter are descriptions of representative cell proliferation conditions.

Lung cancer forms in tissues of the lung, usually in the cells lining air passages. Lung cancer can be divided into two groups accounting for approximately 95% of all lung cancer cases. Types of lung cancer are classified based on the cell size of the tumor, small cell lung cancer (SCLC) and non-small cell lung cancer (NSCLC). The NSCLC includes several types of tumors and is more prevalent. SCLCs are less common but grow quickly and are more likely to metastasize than NSCLCs. Cell lines associated with SCLCs include but are not limited to: DMS 79, DMS 53 and COR L24. About 5% of lung cancers are of rare cell types such as carcinid tumor, lymphoma, or metastatic (cancers from other parts of the body that spread to the lungs. Specific types of lung cancers are as follows. Adenocarcinoma (an NSCLC) is a common type of lung cancer, and cell lines associated with adenocarcinoma include but are not limited to: PC9, 1-87, A594, and PC13. A subtype of adenocarcinoma is called bronchoalveolar cell carcinoma. Squamous cell carcinoma (an NSCLC) is another common type of lung cancer, and squamos cell carcinoma cell lines include but are not limited to: SQ-1, YM21, PC10, and RERF-LC-AI. Other lung cancer cell lines include but are not limited to: A549/ATCC, PC1, PC9, EKVX, HOP-62, HOP-92, NCI-H226, NCI-H23, NCI-H322M, NCI-H460, NCI-H522, DMS 114, SHP-77, LXFL 529, H460, H520, Calu-3, H23, HTB-58, A549, H441, H2170, H1648, H1770, H1819, H1993, H2009, H2087, H2122, and H2347.

The prostate is a gland that produces the liquid component of semen. This cancer is one of the most common cancers in males and is a leading cause of cancer death in men. Prostate cancer cell lines include but are not limited to: PC-3, DU-145, LNCaP and LAPCu.

Leukemia is a cancer that starts in blood-forming tissue such as the bone marrow. Examples include hematopoietic neoplastic disorders, which are diseases involving hyperplastic/neoplastic cells of hematopoietic origin (e.g., arising from myeloid, lymphoid or erythroid lineages, or precursor cells thereof). The diseases can arise from poorly differentiated acute leukemias, e.g., erythroblastic leukemia and acute megakaryoblastic leukemia. Additional myeloid disorders include, but are not limited to, acute promyeloid leukemia (APML), acute myelogenous leukemia (AML) and chronic myelogenous leukemia (CML); lymphoid malignancies include, but are not limited to acute lymphoblastic leukemia (ALL), which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof (see below), peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease with chronic lymphocytic leukemia being the most common type. Cell lines associated with Leukemia include but are not limited to: Karpas 229, SU-DHL-1, SR-786, HUT-78, HH, BC-1L, BC-3L, IM9, Mino, Sp-53, Z138, JM-P1, L-1236, L-428, HD-MyZ, HD-LM2, MDA-E, MDA-V, KM-H2, CCRF-CEM, DND41, DoHH2, NB4, HL60(TB), K-562, MOLT-4, RPMI-8226, SR, P388, and P388/ADR, U-937, KCL22, Jurkat, MAC2A, NALM6, REH-1, SKW3, HSB2, HL60, KG-1 THP-1 and ML-1.

Non-Hodgkin's Lymphoma (NHL) is any of a large group of cancers of the immune system. There are many different types of NHL, which can be divided into aggressive and indolent types and can be classified as either B-cell NHL or T-cell NHL. B-cell NHLs include Burkitt's lymphoma (BL), diffuse large B-cell lymphoma (DLBCL), follicular lymphoma, immunoblastic large cell lymphoma, precursor B-lymphoblastic lymphoma, and mantle cell lymphoma. T-cell NHLs include mycosis fungoides, anaplastic large cell lymphoma, and precursor T-lymphoblastic lymphoma. Lymphomas related to lymphoproliferative disorders following bone marrow or stem cell transplantation are usually B-cell NHLs. NHL cell lines include but at not limited to: CA-46, SKI-DLBL, BS, DOHH1, MAC2A, MF4, AB5, JB7, EBV-BJAB, BC1, BC3, BCBL1, and HBL6, ARH77, Daudi, HS-Sultan, IM-9, Jurkat, SKW3, NALM6, REH-1, MC/CAR, Namalwa, Ramos, Raji, RL, RPMI-1788, RPMI-8226, SKW6.4, WIL2/S, SP2/O-Ag14, Granta-519, SUP-B15, K562, DHL-4 and DHL-7, CRL, and SUD4. AIDS-related NHL, commonly associated with BL and DLBCL, cell lines include but are not limited to: LCL8664, 2F7, BCBL-1 and UMCL01-101.

Colon and Rectal cancer are cancers that located in the large intestine (colon) or the rectum (end of the colon) and are often referred to as “colorectal cancers.” Most colon cancers are adenocarcinomas. There is no single cause of colon cancer, however, nearly all begin with polyps which slowly develop into cancers. Colon Cancer cell lines include but are not limited to: COLO 205, COLO 320DM, HCC-2998, HCT-15, HCT-116, HT29, KM12, SW620, DLD-1 and KM20L2.

Breast cancer forms in tissues of the breast, usually the ducts and lobules. It occurs in both men and women, although male breast cancer is rare. Types of breast cancer include ductal carcinoma in-situ, which is divided into comedo and non-comedo types. Other types of breast cancer include infiltrating ductal and medullary carcinomas, which are common forms of the cancer. Medullary carcinoma: Comprise 15% of breast cancers. Infiltrating lobular forms for breast cancer generally present in the upper outer quadrant of the breast as a subtle thickening and can be bilateral. Microscopically, these tumors exhibit a linear array of cells and grow around the ducts and lobules. Tubular carcinomas present as orderly or well differentiated carcinoma of the breast. Other types of breast cancer include mucinous carcinomas and inflammatory breast cancer, the latter of which is a particularly aggressive type of breast cancer tumor located in the lymphatic and vascular channels. Breast cancer cell lines can include but are not limited to: MDA-MB-486, MCF-7, NCLADR-RES, MDA-MB-231/ATCC, HS 578T, MDA-MB-435, MDA-N, BT-549, T-47D, SUM-52, H184A1, CAMA-1, CAL51, SK-Br-3 and BT-474.

Ovarian cancer is a common cancer in women worldwide. Generally occurring in women over the age of fifty years there are several types of ovarian cancer. The cancer type is classified according to the type of cell from which they originate. Epithelial Ovarian Cancer (EOC) is common and arises from cells covering the surface of the ovaries. Germ Cell Carcinoma arises from cells that form the eggs. There are several types of germ cell carcinomas: teratomas, dysgerminomas, endodermal sinus tumors, embryonal cell carcinoma, choriocarcinoma, polyembriona and mixed germ cell carcinomas. Stromal carcinoma occurs in the connective tissue cells that hold the ovary together and cells that produce estrogen and progesterone. Common types of stromal carcinoma are granulose cell tumors and Stertoli-Leydig cell tumors. Ovarian cancer cell lines include but are not limited to: IGR-OV1, IGROV-1/pt-1, OVCAR-3, OVCAR-4, OVCAR-5, OVCAR-8, SK-OV-3, SK-OV-3TR (MDR), A2008, A2780 and CAOV3.

Renal (kidney) carcinomas are prevalent among the cancers diagnosed worldwide. Kidney cancer includes renal cell carcinoma (adenocarcinoma) and renal pelvis carcinoma (hypernephroma). It also includes Wilms' tumor, which is a type of kidney cancer that usually develops in children under the age of 5. Renal cancer cell lines include but are not limited to: 786-0, A498, ACHN, CAKI-1, RXF-393, SN12C, TK-10, UO-31, RXF-631, SN12K1

Skin cancer is a cancer that begins in the epidermis. There are three different types of skin cancer, two non-melanoma type cancers (i.e., squamos cell carcinoma and basal cell carcinoma) and one melanocyte-type carcinoma generally known as melanoma. Non-Melanoma and Melanoma Skin Cancer cell lines include but are not limited to: A431, RPMI-7591, SCC25, M19-MEL, LOX IMVI, MALME-3M, M14, SK-MEL2, SK-MEL-28, SK-MEL-5, UACC-257 and UACC-62.

Bladder cancer forms in tissues of the bladder and are usually transitional cell carcinomas (TCC). Types of bladder cancers include squamous cell carcinoma and adenocarcinoma. The cells that form squamous cell carcinoma and adenocarcinoma develop in the inner lining of the bladder as a result of chronic irritation and inflammation. Bladder cancer cell lines include but are not limited to: KU-7, UMUC-2, UMUM-3, UMUC-6, PC-3 5637, CAT(wil), EGEN, 253J, BIU87, SCaBER, J28, T24, TCC-SUP, MBT-2, EJ-28, EJ-138, T24 and RT112.

The central nervous system (CNS) is made up of the brain and spinal cord. The brain is made up of nerve cells and tissue and is divided into three major sections: the cerebrum, the cerebellum and the brainstem. The spinal cord is made up of bundles of nerve fibers that begin at the base of the brain and extend about half way down the back connecting the brain with other nerves throughout the body. Cancers of the CNS can occur in any part of the brain or spinal cord. Tumors originating in the brain are called primary brain tumors and are classified according to the kind of cell from which the tumor seems to originate. The most common primary brain tumor in adults comes from cells in the brain called astrocytes that make up the blood-brain barrier and are called gliomas (astrocytoma, anaplastic astrocytoma, or glioblastoma multiforme) and account for 65% of all primary central nervous system tumors. Other types of brain tumors include oligodendroglioma carcinomas, which are from oligodendrocyte cells. Oligodendrocyte cells produce a substance called myelin, which covers the nerves and helps information to travel between the brain and other parts of the body. Ependymoma carcinomas are from Ependyma cells which line the ventricles and aids in the circulation of cerebrospinal fluid. Meningioma carcinomas are from Meninges cells that cover and protect the brain and spinal cord. Lymphoma carcinomas are from lymphocyte cells. Schwannoma carcinomas are from Schwann cells, which produce the myelin that protects the acoustic nerve. Medulloblastoma carcinomas are from primitive neuroectodermal cells or Primitive nerve tumors (PNET). Central Nervous System (CNS) cancer cell lines include but are not limited to: SF-620, SF-268, SF-295, SF-539, SNB-19, SNB-75, U251, SNB-78 and XF-498.

Thyroid cancer is a cancer that forms in the thyroid gland. Four main types of thyroid cancer are papillary (PTC), follicular (FTC), medullary (MTC) and anaplastic thyroid cancer (ATC). Thyroid cancer cell lines include but are not limited to: ARO, FRO, KTC1, KTC2, KTC3, 8305C, 83505C, HTh-104, HTh-112, HTh-7, HTh-74, HTH-83, C-643, JAT-4 and SW-1736.

Pancreatic cancer is another leading cause of cancer deaths. Pancreatic cancer cells are found in the tissues of the pancreas. Nearly 95% of pancreatic cancer arises from the exocrine portion of the organ. The least common exocrine cancer comes from acinar cells. The most common exocrine tumors are adenocarcinomas from ductal cells. Pancreatic cell lines include but are not limited to: ASPC1, MiaPaca2, HS766T, BxPC-3, PCI43, MIAPaCa-2, L36PL, Panc1, Panc203, Panc1005, MPanc-96, XPA3, XPA4, E3LZ10 and E3JD13.

Endometrial Cancer is a cancer that forms in the tissue lining the uterus. Most endometrial cancers are adenocarcinomas. Endometrial cancers cell lines include but are not limited to: HEC-1A, HEC-1B, HHUA, RL95-2, AN3CA, Ishikawa, ECC-1, KLE, SKUT1, and SKUT1B.

Regulatory peptide receptors are overexpressed in numerous human cancers in comparison to their expression in normal tissue adjacent to the neoplasm and/or in its normal tissue of origin. In vitro studies have shown that many cancers can overexpress not only one but several peptide receptors concomitantly. The sub-types of these receptors include, but are not limited to, the somatostatin receptors sst1-sst5, the VIP receptors VPAC1 and VPAC2, the CCK1 and CCK2 receptors, the three bombesin receptor subtypes BB1(NMB receptor), BB2 (GRP receptor) and BB3, and GLP-1 receptors.

Neuroendocrine tumors have often been found to express peptide receptors in varying degrees. Neuroendocrine cancers include several sub-types such as medullary thyroid carcinomas, small cell lung cancers, gastroenteropancreatic tumors, growth hormone pituitary adenomas, pheochromocytomas, paragangliomas, neuroblastomas and parathyroid adenomas.

Neuroendocrine tumors are frequently classified into the following categories: (1) multiple endocrine neoplasia type 1 (MEN1), which are associated with tumors of the parathyroid, pituitary, pancreas, adrenal gland, thyroid, as well as carcinoid tumors, lipomas and angiomas; (2) multiple endocrine neoplasia type 2 (MEN2); (3) carcinoid tumors; (4) islet cell tumors; (5) pheochromocytomas and paragangliomas; and (6) poorly differentiated neuroendocrine tumors, small cell carcinomas other than lung, or atypical lung carcinoids. See NCCN CLINICAL PRACTICE GUIDELINES IN ONCOLOGY™, Neuroendocrine Tumors, V.1.2008, available at http://www.nccn.org/professionals/physician_gls/PDF/neuroendocrine.pdf.

MEN1 tumors include, e.g., gastrinomas, glucagonomas, insulinomas, VIPomas, pancreatic polypeptidomas, somatostatinomas, pituitary tumors, adrenal gland tumors, liver metastases, thymus and bronchial carcinoids, and lipomas. MEN2 tumors include, e.g., medullary thyroid carcinomas, pheochromcytomas and neuromas. Carcinoid tumors include, e.g., thymus and bronchial carcinoid tumors (e.g., small cell carcinoma), gastric tumors, and tumors of the appendix, small bowel, colon and rectum. Islet cell tumors include, e.g., gastrinoma, glucagonomas, insulinomas, VIPomas, pancreatic polypeptidomas, somatostatinomas, nonfunctioning pancreatic tumors. Additional neuroendocrine tumors include, e.g., pheochromocytomas and paragangliomas.

Tumors of the central nervous system have been shown often to express peptide receptors in varying degrees. Nervous system cancers include astrocytomas, meningiomas, schwannomas, medullablastomas and glioblastomas. Tumors of the reproductive system have been shown often to express peptide receptors in varying degrees. Reproductive system tumors include breast carcinomas, endometrial carcinomas, ovarian cancers, leiomyomas, epithelial and stromal tumors, and prostate carcinomas.

The nuclear shape of a cell reflects the internal nuclear structure and processes. A characteristic common to cancer cells is abnormal nuclear shape and the presence of abnormal nucleoli. The abnormalities are so prevalent in cancer cells that they are commonly used as a pathological marker of cell transformation. Central to the nucleoli is the nuclear matrix which is responsible for regulation of cellular processes such as DNA replication and transcription. The nuclear matrix is the framework of the nucleus and includes the peripheral lamins and pore complexes, an internal ribonucleic protein network, and residual nucleoli. The composition of the nuclear matrix is tissue specific and can serve as a marker of each cell and tissue type. Abnormalities include the presence of abnormal nuclear matrix proteins (NMP), abnormal DNA and abnormal RNA and can be unique to certain cell types or cell states. Abnormal expression of nuclear proteins that are strongly associated with cell proliferation including proteins involved in regulation of DNA and RNA synthesis and cellular regulation pathways is also characteristic of cancer cells. This is especially true in prostate, renal, breast, colon, cervical, bladder and head and neck cancers and bladder cancers and where prominent nucleoli are a histologic hallmark.

Cancer from other parts of the body can spread to the other parts of the body and cause secondary tumors through a process called metastasis. The cells of metastatic tumors resemble the cells of the organ from where the tumor started, not necessarily the location where a tumor is ultimately found. For example, if a tumor starts in the breast and spreads to the brain, the cells of the brain tumor will resemble abnormal breast cells, not abnormal brain cells.

Inhibition of cell proliferation can be determined in vitro. Multiple in vitro cell culture techniques and determinations of cell viability and proliferation are known to and selected by the person of ordinary skill in the art. For example, markers for cell viability and cell proliferation include (i) DNA synthesis (e.g., monitoring bromodeoxyuridine incorporation into DNA); (ii) cell cycle progression (e.g., propidium iodide treatment of cells); (iii) cell staining (e.g., Annexin-V and 7-aminoactinomycin (ADD) staining); (iv) DNA fragmentation and caspase cleavage as a measure of apoptosis (e.g., caspase 3 activity and terminal deoxynucleotidyl transferase biotin-dUTP nick end labeling (TUNEL)); and (v) colony formation of cells (e.g., 2-(P-iodophenyl)-3-(p-nitrophenyl)-5-phenyl tetrazolium chloride (INT) staining of tumor cells grown in soft agar). In vivo determinations of cell proliferation inhibition can be determined by administering an agent to a subject and measuring tumor size and disease progression or stabilization, as described in greater detail below.

### Administration of Compositions

The invention also in part provides pharmaceutical compositions comprising at least one therapeutic agent within the scope of the invention as described herein, which optionally may be administered in combination with at least one other compound. The composition may comprise a diluent or other pharmaceutically acceptable excipients. The pharmaceutical composition may be administered in an amount effective to treat a condition associated with aberrant cell proliferation in a subject in need thereof. The subject may be a research animal (e.g., rodent, dog, cat, monkey), optionally containing a tumor such as a xenograft tumor (e.g., human tumor), for example, or may be a human.

The terms “treat” and “treating” as used herein refer to ameliorating, alleviating, lessening, and removing symptoms of a disease or condition. A candidate molecule or compound described herein may be in a therapeutically effective amount in a formulation or medicament, which is an amount that can lead to a biological effect, such as apoptosis of certain cells (e.g., cancer cells), reduction of proliferation of certain cells, or lead to ameliorating, alleviating, lessening, or removing symptoms of a disease or condition, for example. The terms also can refer to reducing or stopping a cell proliferation rate (e.g., slowing or halting tumor growth) or reducing the number of proliferating cancer cells (e.g., removing part or all of a tumor). These terms also are applicable to reducing a titre of a microorganism in a system (i.e., cell, tissue, or subject) infected with a microorganism, reducing the rate of microbial propagation, reducing the number of symptoms or an effect of a symptom associated with the microbial infection, and/or removing detectable amounts of the microbe from the system. Examples of microorganism include but are not limited to virus, bacterium and fungus.

In certain embodiments, a therapeutic agent may treat a condition by specifically inhibiting proliferation of cells associated with the condition to be treated. “Specifically inhibiting” or “specifically targeting” as used herein refers to inhibiting proliferation of cells associated with the condition to be treated more than inhibiting proliferation of “normal” cells. An example of a compound that specifically inhibits cells associated with a cell proliferation condition is compound TA1-1B, which inhibits leukemia cells without inhibiting normal bone marrow cells. Cell proliferation may be inhibited in a system comprising a cell, tissue or subject.

Agents described herein can result in apoptosis, and can thereby inhibit cell proliferation by resulting in the death of proliferating cells. As used herein, the term “apoptosis” refers to an intrinsic cell self-destruction or suicide program. In response to a triggering stimulus, cells undergo a cascade of events including cell shrinkage, blebbing of cell membranes and chromatic condensation and fragmentation. These events culminate in cell conversion to clusters of membrane-bound particles (apoptotic bodies), which are thereafter engulfed by macrophages.

The amount of the therapeutic agent, and optionally one or more combination agents, to be administered will vary with the route of administration, the condition of the subject, other treatments being administered to the subject, and other parameters. The therapeutic agents of the invention may, of course, cause multiple desired effects; and the amount of modulator to be used in combination with the therapeutic agent should be an amount that increases one or more of these desired effects.

For administration to animal or human subjects, the appropriate dosage of the therapeutic agent sometimes is 0.01-15 mg/kg, preferably 0.1-10 mg/kg. Dosage levels are dependent on the nature of the condition, drug efficacy, the condition of the patient, the judgment of the practitioner, and the frequency and mode of administration; however, optimization of such parameters is within the ordinary level of skill in the art.

Similarly, the dosage of another compound used in combination with the therapeutic agent sometimes is between about 0.01-15 mg/kg, and can be about 0.1-10 mg/kg. Another agent used in combination with a therapeutic agent described herein may be separately active for treating a cancer. For combination therapies described above, when used in combination with a therapeutic agent, the dosage of another agent sometimes will be two-fold to ten-fold lower than the dosage required when the other agent is used alone to treat the same condition or subject. Determination of a suitable amount of the agent for use in combination with a therapeutic agent is readily determined by methods known in the art.

Any suitable formulation of the therapeutic agent can be prepared for administration. Any suitable route of administration may be used, including but not limited to oral, parenteral, intravenous, intramuscular, nasal, transdermal, topical and subcutaneous routes, and the like. In many embodiments the therapeutic agent may be administered orally. Depending on the subject to be treated, the mode of administration, and the type of treatment desired—e.g., prevention, prophylaxis, therapy; the compounds are formulated in ways consonant with these parameters. The formulation often is prepared according to the selected route of administration as known by the person of ordinary skill in the art. Preparation of suitable formulations for each route of administration are known in the art. A summary of such formulation methods and techniques is found in Remington's Pharmaceutical Sciences, latest edition, Mack Publishing Co., Easton, Pa., which is incorporated herein by reference. The formulation of each substance or a combination of two or more substances will generally include a diluent as well as, in some cases, adjuvants, buffers, preservatives and the like. Thus, provided herein are pharmaceutical compositions comprising a therapeutic agent and a pharmaceutically acceptable excipient. The substances to be administered can be administered also in liposomal compositions or as microemulsions.

For injection, formulations can be prepared in conventional forms as liquid solutions or suspensions or as solid forms suitable for solution or suspension in liquid prior to injection or as emulsions. Suitable excipients include, for example, water, saline, dextrose, glycerol and the like. Such compositions may also contain amounts of nontoxic auxiliary substances such as wetting or emulsifying agents, pH buffering agents and the like, such as, for example, sodium acetate, sorbitan monolaurate, and so forth.

Various sustained release systems for drugs have also been devised, and can be applied to compounds of the invention. See, for example, U.S. Pat. No. 5,624,677, the methods of which are incorporated herein by reference.

Systemic administration may also include relatively noninvasive methods such as the use of suppositories, transdermal patches, transmucosal delivery and intranasal administration. Oral administration is also suitable for compounds of the invention. Suitable forms include syrups, capsules, tablets, as is understood in the art.

The therapeutic agent may be administered in conjunction with another agent, and the agents may be administered separately or together. When administered together, they may be in separate dosage forms, or they may be combined into a single combination drug.

A combination agent, when utilized, is administered in an amount that is effective to enhance a desired effect of the therapeutic agent. An amount is “effective to enhance a desired effect of the therapeutic agent”, as used herein, if it increases by at least about 25% at least one of the desired effects of the therapeutic agent alone. Preferably, it is an amount that increases a desired effect of the therapeutic agent by at least 50% or by at least 100% (i.e., it doubles the effective activity of the therapeutic agent.) In some embodiments, it is an amount that increases a desired effect of the therapeutic agent by at least 200%.

The amount of a combination agent that increases a desired effect of a therapeutic agent may be determined using in vitro methods, such as cell proliferation assays. The therapeutic agents of the invention are useful to counter hyperproliferative disorders such as cancer, thus they reduce cell proliferation. Thus, for example, a suitable amount of a combination agent could be the amount needed to enhance an antiproliferative effect of a therapeutic agent by at least 25% as determined in a cell proliferation assay.

The combination agent used in the present invention enhances at least one desired effect produced by the therapeutic agent it is used with, thus the combinations of the invention provide a synergistic effect, not merely an additive effect. The combination agents themselves are at times useful for treating the same types of conditions, and thus may also have some direct effect in such assays. In that event, the “amount effective to increase a desired effect” must be a synergistic enhancement of the activity of the therapeutic agent that is attributable to enhancement by the combination agent of an effect of the therapeutic agent, rather than a simple additive effect that would be expected with separate administration of the two materials. In many cases, the combination agent can be used in an amount (concentration) that would not be expected to have any apparent effect on the treated subject or the in vitro assay, so the increased effect achieved with the combination is directly attributable to a synergistic effect.

The following examples illustrate and do not limit the invention.

### EXAMPLE 1

### Inhibition of Cells Associated with Aberrant Cell Proliferation Conditions

Inhibition of cell proliferation is determined in human tumor cell lines. Innoculation density and doubling times for specific cell lines are listed at World Wide Web URL “dtp.nci.nih.gov/docs/misc/common_files/cell_list.html.”

### Materials

PBS: Mediatech, catalog #21-031-CV, Dulbecco's Phosphate Buffered Saline, without calcium and magnesium

MTT: Sigma, catalog #M2128

Velcade: R&D Healthcare, Inc., San Diego, Calif. (www.rndhealthcare.com)

Etoposide: Sigma, catalog #E1383

Taxol: Sigma, catalog #T7191

DMSO: Sigma, catalog #D4540, Dimethylsulfoxide

Cells: NCI 60 panel, listed below

Compound: Compound TA1-1B, 16.5 mM stock in PBS

### Procedure

1. Plate adherent cells the day before starting the experiment in 180 μl growth media (see list hereafter).
2. Day of experiment, count and plate suspension cells in 180 μl growth media (see list on next page).
3. Make 10× Compound, vehicle, and chemotherapeutic cocktail
   a. Compound (1× to 10×)
      i. 10 μM Compound to 100 μM
      ii. 3 μM Compound to 30 μM
      iii. 1 μM Compound to 10 μM
      iv. 0.3 μM Compound to 3 μM
      v. 0.1 μM Compound to 1 μM
   b. Vehicle
      i. PBS, equivalent to the volume used for 10 μM Compound (1× is ˜0.06% PBS)
   c. Chemotherapeutic cocktail (1× to 10×):
      i. 1 μM Velcade to 10 μM
      ii. 100 μM Etoposide to 1 mM
      iii. 20 μM Taxol to 200 μM
4. Add 20 μl of the 10× compound/vehicle/chemotherapeutic cocktail to cells.
5. Incubate at 37° C., 5% CO₂ for 48 hours.
6. Aspirate media. For suspension cells, spin plate at 1500 RPM for 10 minutes. Use a multichannel pipettor to slowly remove media.
7. Add 200 μl of Thiazolyl Blue Tetrazolium Bromide (MTT) to each well: 0.863 mg/ml MTT in growth media.

### Cell Lines Used

Adherent cells (20,000 cells/well): SKMEL2; SKMEL5; SKMEL28; TK10; OVCAR4; UO-31; OVCAR8; LOXIMVI; COLO205; UACC-257; NCI-H23; SF-539; SF-268; M14; HOP-92; OVCAR5; SNB-75; SN12C; KM12; PC3; A459; MCF-7; NCI-H522; HCC-2998; CAKI-1; HT29; HCT116; SK-OV-3; DU-145; MDA-MB-231; MDA-MB-435; MALME-3M; SW-620; ADR-RES; EKVX; NCI-H226; NCI-H322M; RXF-393; IGR-OV1; OVCAR3; ACHN; BT-549; T-47D and HS-578T.

Adherent cells (12,000 cells/well): SF-295; UACC-62; U251; HCT15; SNB-19; NCI-H460; 786-0; A498 and HOP-62.

Suspension cells (50,000 cells/well): MOLT-4; RPMI-8226; CCRF-CEM; SR and HL60.

Suspension cells (25,000 cells/well): K562.

Proliferation of several cancer cells was inhibited by compound TA1-1B (which is a particular mixture of the isomers represented by TA1-1A), as shown in the following table (Table 1). The value EC₅₀ is the amount of Compound TA1-1B that results in 50% inhibition of proliferation of the indicated cell type. This broad spectrum of activity against many cell lines demonstrates that TA1-1B is useful to treat a variety of cancers.

TABLE 1

| Cell | Organ | EC50 |
|---|---|---|
| NCI/ADR-RES | Breast | 1.624 |
| BT-549 | Breast | 1.799 |
| HS 578T | Breast | 1.941 |
| MCF7 | Breast | 3.461 |
| MDA-MB-435 | Breast | 3.632 |
| T-47D | Breast | 6.559 |
| SF-268 | CNS | 1.231 |
| SF-295 | CNS | 4.269 |
| SF-539 | CNS | 1.107 |
| SNB-19 | CNS | 4.631 |
| SNB-75 | CNS | 2.31 |
| U251 | CNS | 2.681 |
| COLO 205 | Colon | 0.7069 |
| HCC-2998 | Colon | 2.541 |
| HCT-15 | Colon | 1.507 |
| KM12 | Colon | 3.055 |
| A3 | Leukemia | 3.63 |
| CCRF-CEM | Leukemia | 0.6296 |
| CCRF-CEM | Leukemia | 1.22 |
| D1-1 | Leukemia | 4.16 |
| GDM-1 | Leukemia | 1.26 |
| HL-60(TB) | Leukemia | 1.65 |
| I 9.2 | Leukemia | 3.69 |
| J45-01 | Leukemia | 1.89 |
| Jgamma-1 | Leukemia | 1.72 |
| Jurkat | Leukemia | 1.21 |
| K-562 | Leukemia | 3.125 |
| K-562 | Leukemia | 1.52 |
| Kasumi-1 | Leukemia | 2.05 |
| KG-1 | Leukemia | 3.97 |
| Ku-812 | Leukemia | 4.19 |
| MEG-01 | Leukemia | 6.00 |
| MOLT-3 | Leukemia | 1.14 |
| MOLT-4 | Leukemia | 1.212 |
| MOLT4 | Leukemia | 1.22 |
| MV-4-11 | Leukemia | 5.81 |
| P116 | Leukemia | 1.23 |
| Reh | Leukemia | 1.32 |
| RPMI-8226 | Leukemia | 1.367 |
| RS4-11 | Leukemia | 0.55 |
| SR | Leukemia | 0.9144 |
| SR | Leukemia | 1.30 |
| TF-1 | Leukemia | 5.46 |
| THP-1 | Leukemia | 3.18 |
| Daudi | Lymphoma | 1.29 |
| H9 | Lymphoma | 2.238 |
| HH | Lymphoma | 4.04 |
| HuT102 | Lymphoma | 1.05 |
| Hut78 | Lymphoma | 3.3 |
| JM1 | Lymphoma | 2.228 |
| Karpas299 | Lymphoma | 3.92 |
| MC116 | Lymphoma | 3.871 |
| Namalwa | Lymphoma | 1.42 |
| Raji | Lymphoma | 1.88 |
| Ramos | Lymphoma | 1.33 |
| RS1184 | Lymphoma | 3.247 |
| ST486 | Lymphoma | 1.279 |
| U266B1 | Lymphoma | 1.616 |
| U937 | Lymphoma | 4.54 |
| RPMI-6666 | Lymphoma | 1.593 |
| NC37 | Lymphoma | 1.261 |
| SUDHL4 | Lymphoma | 1.339 |
| HT | Lymphoma | 2.44 |
| DOHH2 | Lymphoma | 1.564 |
| Hs602 | Lymphoma | 1.282 |
| NK92MI | Lymphoma | 1.296 |
| M14 | Melanoma | 4.982 |
| MALME-3M | Melanoma | 5.09 |
| UACC-257 | Melanoma | 5.103 |
| UACC-62 | Melanoma | 4.88 |
| HOP-92 | Non Small Cell Lung | 1.805 |
| HOP-92 | Non Small Cell Lung | 1.961 |
| EKVX | Non Small Cell Lung | 5.674 |
| NCI-H322M | Non Small Cell Lung | 4.283 |
| NCI-H460 | Non Small Cell Lung | 4.009 |
| NCI-H522 | Non Small Cell Lung | 1.504 |
| IGROV1 | Ovarian | 4.437 |
| OVCAR-3 | Ovarian | 4.28 |
| OVCAR-4 | Ovarian | 2.959 |
| OVCAR-5 | Ovarian | 2.17 |
| PC-3 | Prostate | 4.96 |
| 786-0 | Renal | 4.345 |
| SN12C | Renal | 4.156 |
| TK-10 | Renal | 3.327 |
| UO-31 | Renal | 2.385 |

TA1-1B binds to several receptors, many of which are established cancer treatment targets, as shown in the following table (Table 2):

TABLE 2

| | % inhibition | |
|---|---|---|
| Receptor | @ 1 uM | Name |
| NK₂ | 107 | Neurokinin NK2 receptor |
| M₁ | 98 | Muscarinic receptor |
| CCK_{B}/CCK₂ | 93 | Cholecystokinin receptor |
| M₂ | 92 | Muscarinic receptor |
| M₄ | 87 | Muscarinic receptor |
| σ (non-selective) | 67 | sigma receptors |
| BZD (peripheral) | 65 | peripheral Benzodiazepine |
| | | receptor |
| CCK_{A}/CCK₁ | 64 | Cholecystokinin receptor |
| sst (non-selective) | 62 | Somatostatin receptor |
| A_{2A} | 60 | Adenosine A2A receptors |
| κ-opioid (KOP) | 52 | opioid receptor |
| 5-HT_{1B} | 49 | 5-hydroxytryptamine |
| | | (serotonin) receptor 1B |
| H₂ | 48 | H2-receptor |

### EXAMPLE 2

### Compound TA1-1B is Not Toxic to Bone Marrow Cells

Rats were treated with 10 milligrams per kilogram of Compound TA1-1B daily for five (5) days. Twenty-four (24) hours after the last dose the bone marrow was isolated, and bone marrow cells (BMCs) were tested for RNA synthesis activity and cell viability

The following process was utilized to measure total RNA synthesis. For animals treated with compound TA1-1B, BMCs were isolated and plated overnight at 100,000 cells per mL. Next day cells were incubated for one hour with 5 μCi of [³H]-uridine. To measure total RNA synthesis, total RNA from treated cells was isolated with a RNease kit (QIAGEN), levels of total RNA were assessed with Ribogreen reagent (Invitrogen) and the newly synthesized tritiated RNA was measured in a scintillation Counter (Perkin Elmer) in a tritium channel.

The following process was utilized to measure cell viability, which involves the use of an Alamar Blue dye. Cells are counted using a hemocytometer, and 4,000-5,000 cells (per well) in 100 microliter of medium are seeded into wells 96-well plate. Twenty microliters of Alamar Blue reagent (stored at 4° C.) is added to each well and the cells are incubated for four (4) hours at 37° C., 5% CO₂ in a humidified incubator. Fluorescence is recorded at an excitation wavelength of 544 nm and emission wavelength of 590 nm using a microplate reader. Fluorescence of non-reduced dye is detected and effects of drug treatment on BMC viablity is determined.

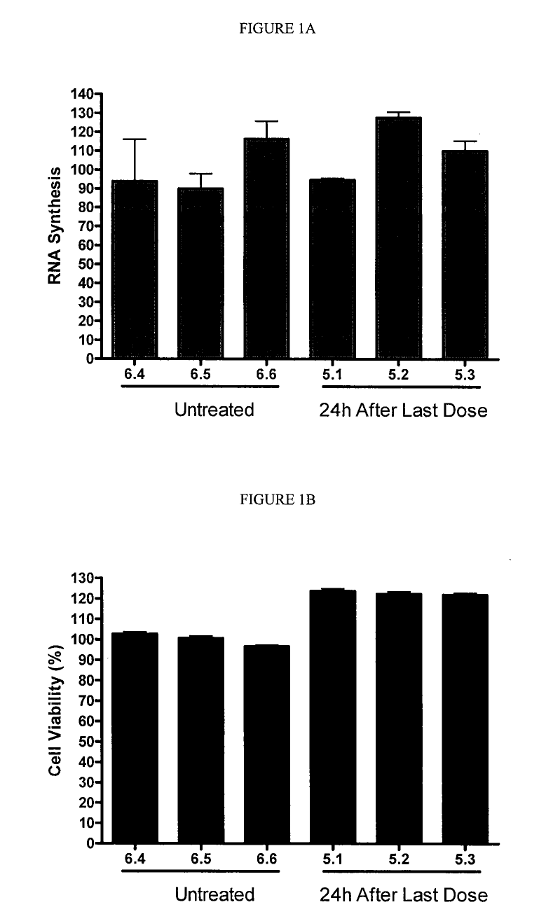

FIG. 1A and FIG. 1B show effects of compound TA1-1B on RNA synthesis and cell viability, respectively, in bone marrow cells. These results show the compound is not toxic to the BMCs. Because the compound, however, is toxic to leukemia cells (see Example 1), these results suggest the compound specifically targets leukemia over “normal” cells.

### EXAMPLE 3

### Compound TA1-1B Permeates the Blood-Brain Barrier

In vivo experiment protocols were approved by the Animal Use and Care Committee. Female NCr nu/nu mice were purchased from Taconic Farms and group housed in a ventilated rack system on a 12/12 light cycle. All housing materials and water were autoclaved prior to use. The mice were fed ad libitum with gamma-irradiated laboratory chow and acidified water. Animals were handled under laminar-flow hoods. Animals were inoculated subcutaneously in the right flank with 5×10̂6 HCT116 cells. Tumors were monitored twice weekly and then daily as they approached the appropriate size for study. On Day 1 of the study, the animals were randomized into n=3 treatment groups with group average tumor sizes of 109 mm³.

Tumor size was calculated using the formula (1×w²)/2

Tumor Volume (mm³)=(1×w²)/2

where w=width and 1=length in mm of the tumor. Tumor weight was estimated with the assumption that 1 mg is equivalent to 1 mm³ of tumor volume. Mice were dosed with compound TA1-1B at 25.0 mg/kg (single IV dose). At fifteen min and two hour time points, plasma, brain and tumor samples were collected from terminal mice and frozen. It was determined that the tumor contained 0.86 μM, plasma contained 0.74 μM and brain contained 1.47 μM of the therapeutic agent after 15 min post-dose, and the tumor contained 1.6 μM, plasma contained 0.41 μM and brain contained 0.8 μM of the therapeutic agent after the two hour post-dose.

### EXAMPLE 4

### Compound TA4-1A Permeates the Blood-Brain Barrier

In vivo experiment protocols were approved by the Animal Use and Care Committee. Female NCr nu/nu mice were purchased from Taconic Farms and group housed in a ventilated rack system on a 12/12 light cycle. All housing materials and water were autoclaved prior to use. The mice were fed ad libitum with gamma-irradiated laboratory chow and acidified water. Animals were handled under laminar-flow hoods. Animals were inoculated subcutaneously in the right flank with 5×10̂6 HCT116 cells. Tumors were monitored twice weekly and then daily as they approached the appropriate size for study. On Day 1 of the study, the animals were randomized into n=3 treatment groups with group average tumor sizes of 109 mm³.

Tumor size was calculated using the formula (1×w²)/2

Tumor Volume (mm³)=(1×w²)/2

where w=width and 1=length in mm of the tumor. Tumor weight was estimated with the assumption that 1 mg is equivalent to 1 mm³ of tumor volume. Mice were daily dosed with compound TA4-1A at 12.5 mg/kg (IV) during 14 days. A wash-out period ensued for four days with no dosing, and the last dose was administered after the wash-out period (day 19) at 12.5 mg/kg. At one, two and twenty-four hour time points plasma, brain and tumor samples were collected from terminal mice and frozen. It was determined that the tumor contained 35%, plasma contained 64% and brain contained 1% of the therapeutic agent after the one hour time point, and the tumor contained 74%, plasma contained 24% and brain contained 2% of the therapeutic agent after the two hour time point. At the 24 hour time point, tumor contained 17%, plasma contained 49% and brain contained 34% of the therapeutic agent. After an eight hour exposure to the agent in female and male rats, approximately 3-4% of the agent was available in the brain as compared to the amount in plasma. These results demonstrate that compound TA4-1A can permeate the blood-brain barrier to some extent and can enter the brain, and therefore is useful for treating conditions associated with aberrant cell proliferation in the brain. Examples of cell lines and particular brain cancers are described herein.

### EXAMPLE 5

### Compound TA4-1B Binds Red and White Blood Cells

Twenty five mL healthy donor whole blood was collected in the presence of heparin. Human whole blood was added with Compound TA1-1B to reach final concentration of 3 uM. Whole blood was gently mized and incubated at 37° C. for 4 hrs. At the end of the incubation 0.5 mL blood was collected and centrifuge at 500×g to separate the platelet-rich plasma for further analysis.

In order to isolate white blood cells, 5 ml of treated blood was subjected to Ficoll density gradient centrifugation. At the end of the centrifugation formed buffy coat containing most of the white blood cells and Ficolle fractions containing red blood cells and granulocites were collected separately. Obtained white cell fraction was washed with 5 ml of isotonic phosphate buffer and saved for further analysis. Obtained fractions were subjected to Compound TA1-1B quantitative analysis using LC-MS/MS. Fractions obtained from untreated whole blood were used as corresponding biological matrices in LC-MS/MS based bioanalysis.

Table 3 shows the distribution of compound TA1-1B in different fractions. These results indicate the compound is capable to bind human red and white cells at high extend. The most part of added compound (93.7%) was found in blood formed elements than in plasma indicating to a potentially high volume of distribution and good tissue penetration of compound TA1-1B.

TABLE 3

| | | Volume | Amount | |
|---|---|---|---|---|
| Fraction | Conc. (uM) | (mL) | (nmole) | % |
| Plasma | 0.37 | 2.5 | 0.92 | 6.2 |
| White cells | 81.79 | 0.022 | 1.8 | 12.2 |
| Red cells | 4.85 | 2.478 | 12.0 | 81.5 |
| Total | | | 14.7 | 100 |
| Mass balance | 2.95 uM | 5 | 14.7 | |

### EXAMPLE 6

### Compound TA4-1B Penetrates Different Tissues in Rats

Five groups (three animals per group) of male Sprague Dawley rats were assigned for in vivo pharmacokinetic study.
Group-1 animals were dosed intravenously with compound TA1-1B at 7.5 mg/kg and after 2 hr post-dose animals were terminated and whole blood, plasma, brain, bone marrow and adrenal gland collected.
Group-2 animals were dosed intravenously with compound TA1-1B at 7.5 mg/kg for five consecutive days. After 2 hr of the last dose (98 hr) animals were terminated and whole blood, plasma, brain, bone marrow and adrenal gland collected.
Group-3 animals were dosed intravenously with compound TA1-1B at 7.5 mg/kg for five consecutive days. After 26 hr of the last dose (122 hr) animals were terminated and whole blood, plasma, brain, bone marrow and adrenal gland collected.
Group-4 animals were dosed intravenously with compound TA1-1B at 7.5 mg/kg for five consecutive days. After 50 hr of the last dose (146 hr) animals were terminated and whole blood, plasma, brain, bone marrow and adrenal gland collected.
Group-5 animals were dosed intravenously with vehicle, terminated and whole blood, plasma, brain, bone marrow and adrenal gland collected. Tissues obtained from this group of animals were used for corresponding matrix preparation in subsequent bioanalysis.

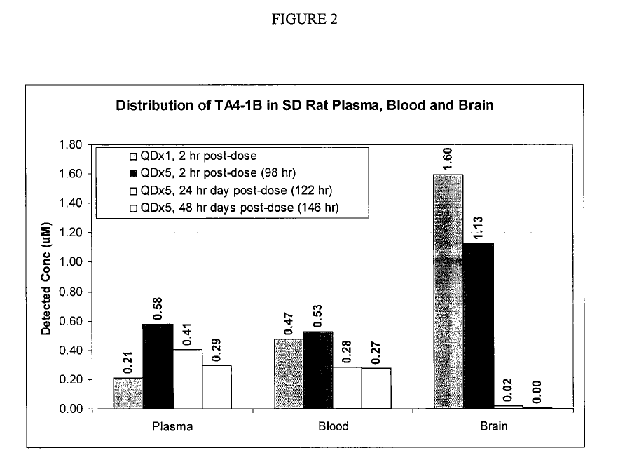

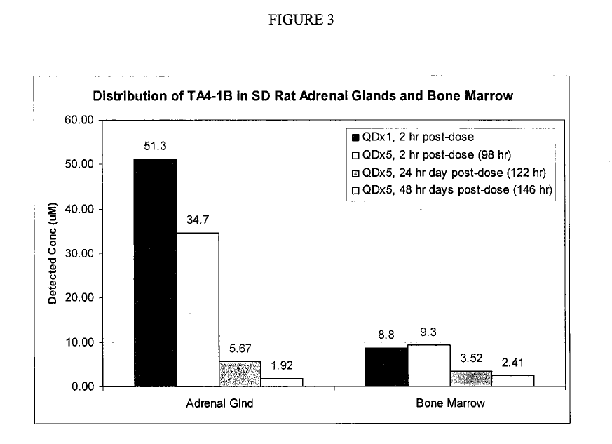

The compound TA1-1B concentration determined in the brain tissue was significantly higher than that measured in plasma or whole blood (1.6, 0.2 and 0.5 respectively). TA1-1B quickly (in 2 hr) associates with rat brain tissue, and almost completely eliminates over 48 hr post-dose (FIG. 2). Adrenal glands and bone marrow tissues showed the highest exposures of TA1-1B. After administration the compound TA1-1B quickly reaches those tissues (after 2 hr) and subsequently gets cleared. Compound TA1-1B retains longer in adrenal glands and bone marrow than in brain (FIG. 3)

### EXAMPLE 7

### Receptor Binding and Functional Data for TA1-1B on Exemplary Receptors

The binding IC₅₀, functional effect, and functional EC₅₀ of TA1-1B on exemplary receptors was determined. Response for CCK1 was noted at a concentration of 1 μM, while other responses were noted at a concentration of 10 μM. Representative receptor data is provided in Table 4.

TABLE 4

| | Binding | Functional | Functional | Functional |
|---|---|---|---|---|
| Receptor | IC₅₀ | Effect | EC₅₀ | K_{B} |
| CCK1/CCKA | 640 nM | Antagonist | 1.6 μM | 340 nM |
| CCK2/CCKB | N.C. | Antagonist | 9.0 μM | 400 nM |
| SST1 | 2.8 μM | Agonist | 4.3 μM | |
| SST4 | >10 μM | Agonist | 9.9 μM | |
| SST5 | >10 μM | Agonist | 4.7 μM | |

### EXAMPLE 8

### Exemplary Embodiments of the Invention

Some representative embodiments of the invention are set forth hereafter, but are not to be taken as limiting the scope of the invention as described herein.

A1. A method for inhibiting proliferation of cells by contacting the cells with a compound having a structure described herein.

A2. The method of embodiment Al, wherein the cells are from a cancer of the breast, blood, colon, rectum, colorectum, lymph system, lymph node, central nervous system, lung, ovary, kidney, skin or prostate.

A3. The method of embodiment A1 or A2, wherein the compound has a structure of formula TA1-1A or TA1-1B.

A4. The method of embodiment A3, wherein the cancer of the blood is a leukemia.

A4′. The method of embodiment A2, wherein the cancer of the central nervous system is a brain cancer.

A4″. The method of embodiment A4′, wherein the brain cancer is a gliomablastoma or a medulloblastoma.

A5. The method of embodiment A4, wherein the leukemia is chronic lymphocytic leukemia (CLL).

A6. The method of embodiment A1 or A2, wherein the compound has a structure of formula TA4-1A.

A7. The method of embodiment A6, wherein the cancer of the central nervous system is a brain cancer.

A8. The method of embodiment A7, wherein the brain cancer is a gliomablastoma or a medulloblastoma.

A9. The method of any one of embodiments A1-A8, wherein the cells are cultured in vitro.

B1. A method for treating a disorder resulting from aberrant cell proliferation in a subject, which comprises administering a therapeutically effective amount of a pharmaceutical composition comprising a compound having a structure described herein, whereby the disorder is treated.

B2. The method of embodiment B1, wherein the disorder is a cancer of the breast, blood, colon, central nervous system, lung, ovary, kidney, skin or prostate.

B3. The method of embodiment B1 or B2, wherein the compound has a structure of formula TA1-1B.

B4. The method of embodiment B3, wherein the cancer of the blood is a leukemia.

B5. The method of embodiment B4, wherein the leukemia is chronic lymphocytic leukemia (CLL).

B6. The method of embodiment B1 or B2, wherein the compound has a structure of formula TA4-1A.

B7. The method of embodiment B3 or B6, wherein the cancer of the central nervous system is a brain cancer.

B8. The method of embodiment B7, wherein the brain cancer is a gliomablastoma or a medulloblastoma.

C1. A method for inhibiting proliferation of cancer cells, comprising contacting a cancer cell with an effective amount of a compound having the formula:

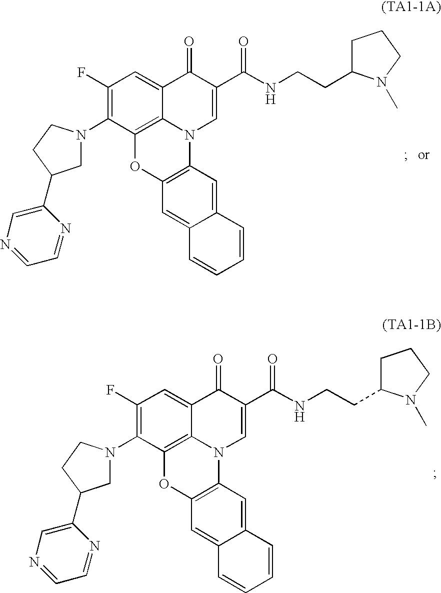

or a pharmaceutically acceptable salt, ester or prodrug thereof, or a specific isomer or mixture of isomers thereof.

C2. The method of embodiment C1, wherein said cancer cells are selected from the group consisting of leukemia cells, lymphoma cells, breast cancer cells, lung cancer cells, central nervous system cancer cells, skin cancer cells, ovarian cancer cells, prostate cancer cells, renal cancer cells, colorectal cancer cells, liver cancer cells, pancreatic cancer cells, adrenal gland cancer cells, thymic cancer cells, lymph node cancer cells, stomach cancer cells, appendix cancer cells, small bowel cancer cells, head and neck cancer cells, heart cancer cells, pituitary gland cancer cells, parathyroid gland cancer cells, and thyroid gland cancer cells.

C3. The method of embodiment C1 or C2, wherein said cancer cells are central nervous system cancer cells.

C4. The method of embodiment C2 or C3, wherein said central nervous system cancer cells are brain cancer cells.

C5. The method of embodiment C4, wherein said brain cancer cells are gliomablastoma cells or medulloblastoma cells.

C6. The method of any one of embodiments C1-C5, wherein the compound has the formula (TA1-1B), or a pharmaceutically acceptable salt thereof.

C7. The method of embodiment C1 or C2, wherein said cancer cells are leukemia cells.

C8. The method of embodiment C7, wherein said leukemia cells are chronic lymphocytic leukemia (CLL) cells and said compound has the formula (TA1-1B), or a pharmaceutically acceptable salt thereof.

C9. The method of embodiment C1, wherein said cancer cells are cultured in vitro.

C10. A method for inhibiting proliferation of a tumor overexpressing one or more peptide receptors, comprising administering to a subject in need thereof a therapeutically effective amount of a compound having formula (TA1-1):

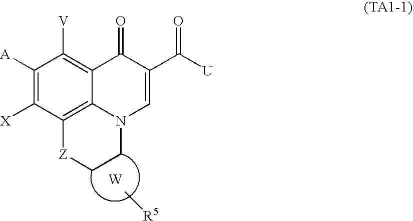

and pharmaceutically acceptable salts, esters and prodrugs thereof;

wherein V is H, halo, or NR¹R²;

A is H, fluoro, or NR¹₂;

Z is O, S, NR¹ or CH₂;

U is OR² or NR¹R²;

X is OR², NR¹R², halo, azido, or SR²;

n is 1-3;

wherein in NR¹R², R¹ and R² may form a double bond or a ring, each of which is optionally substituted;

R¹ is H or a C₁₋₆ alkyl;

R² is H or a C₁₋₁₀ alkyl or C₂₋₁₀ alkenyl optionally containing one or more non-adjacent heteroatoms selected from N, O, and S, and optionally substituted with a carbocyclic or heterocyclic ring; or R² is an optionally substituted heterocyclic ring, aryl or heteroaryl;

R⁵ is a substituent at any position on W; and is H, OR², C₁₋₆ alkyl, C₂₋₆ alkenyl, each optionally substituted by halo, ═O or one or more heteroatoms; or R⁵ is an inorganic substituent; and

W is an optionally substituted aryl or heteroaryl, which may be monocyclic or fused with a single or multiple ring and optionally containing a heteroatom;

or a compound having formula (TA1-2):

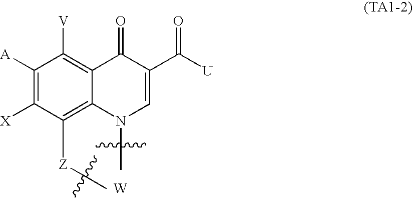

and pharmaceutically acceptable salts, esters and prodrugs thereof;

wherein V, A, X, Z and U are as defined in formula TA1-1, and W is selected from the group consisting of

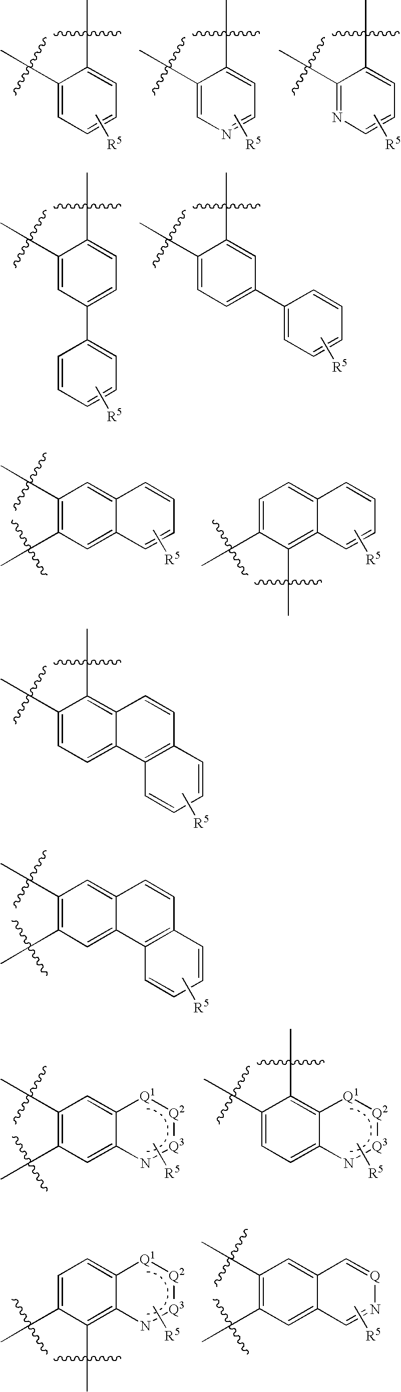

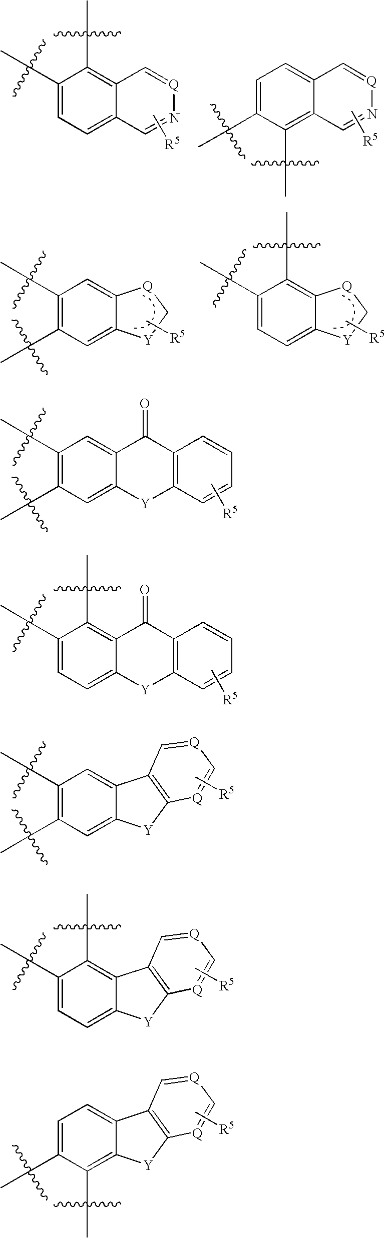

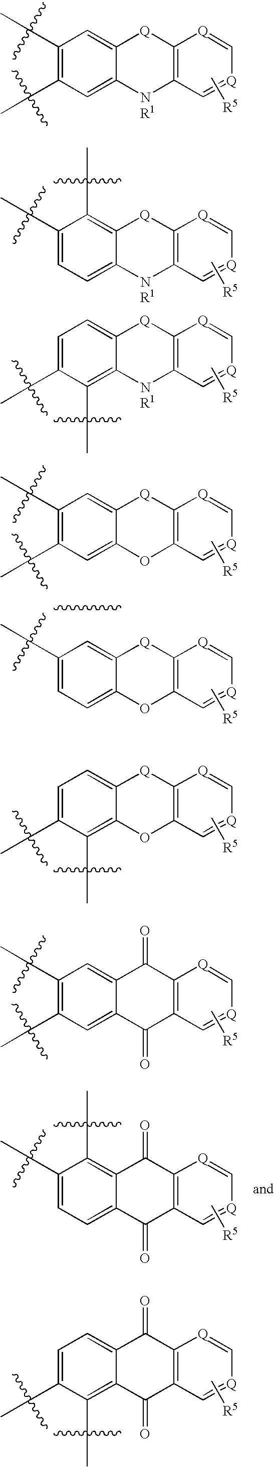

wherein Q, Q¹, Q², and Q³ are independently CH or N;

Y is independently O, CH, ═O or NR¹; and

R⁵ is as defined in formula (TA1-1);

whereby tumor proliferation is inhibited.

C11. The method of embodiment C10, wherein said peptide receptor is selected from the group consisting of the somatostatin receptors sst1-sst5, the VIP receptors VPAC1 and VPAC2, the CCK1 and CCK2 receptors, the bombesin receptor subtypes BB1(NMB receptor), BB2 (GRP receptor) and BB3, and GLP-1 receptors.

C12. The method of embodiment C10 or C11, wherein said tumor is a neuroendocrine tumor selected from the group consisting of multiple endocrine neoplasia type 1 (MEN1), multiple endocrine neoplasia type 2 (MEN2), carcinoid tumors, islet cell tumors, pheochromocytomas and paragangliomas.

C13. The method of embodiment C10, C11 or C12, wherein said tumor is a neuroendocrine tumor selected from the group consisting of medullary thyroid carcinomas, small-cell lung cancers, gastrointestinal stromal tumors (GIST), gastroenteropancreatic tumors (GEP NETs), paragangliomas, pheochromocytomas, exocrine pancreatic cancers, Ewing's sarcomas, adrenal tumors, growth hormone pituitary adenomas, nonfunctioning pituitary adenomas, parathyroid adenomas, gastrinomas, glucagonomas, insulinomas, VIPomas, adrenal tumors, gut carcinoids, ileal carcinoids, and bronchial carcinoids.

C14. The method of embodiment C10 or C11, wherein said tumor is a tumor of the central nervous system selected from the group consisting of astrocytomas, meningiomas, schwannomas, medulloblastomas and glioblastomas.

C15. The method of embodiment C10 or C11, wherein said tumor is a tumor of the reproductive system selected from the group consisting of breast carcinomas, endometrial carcinomas, leiomyomas, ovarian cancers, epithelial and stromal tumors, and prostate carcinomas.

C16. The method of any one of embodiments C10-C15, wherein the compound has the formula:

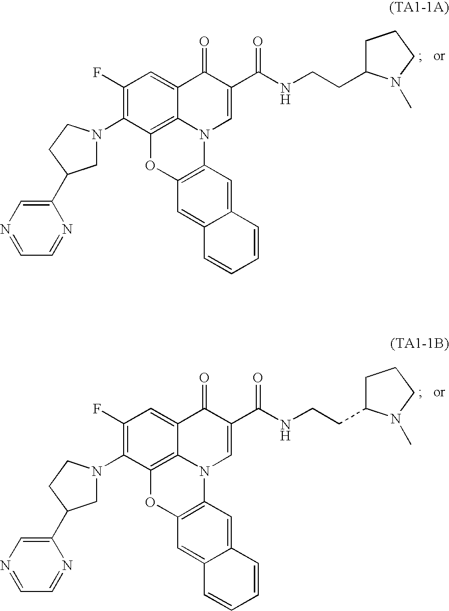

or a pharmaceutically acceptable salt, ester or prodrug thereof, or a specific isomer or mixture of isomers thereof.

C17. The method of embodiment C16, wherein the compound has the formula (TA1-1B), or a pharmaceutically acceptable salt thereof.

C18. A method for treating a disorder resulting from aberrant cell proliferation, comprising administering to a subject in need thereof a therapeutically effective amount of a compound having formula (TA1-1):

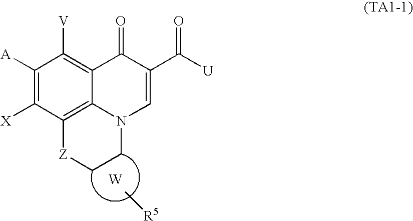

and pharmaceutically acceptable salts, esters and prodrugs thereof;

wherein V is H, halo, or NR¹R²;

A is H, fluoro, or NR¹₂;

Z is O, S, NR¹ or CH₂;

U is OR² or NR¹R²;

X is OR², NR¹R², halo, azido, or SR²;

n is 1-3;

wherein in NR¹R², R¹ and R² may form a double bond or a ring, each of which is optionally substituted;

R¹ is H or a C₁₋₆ alkyl;

R² is H or a C₁₋₁₀ alkyl or C₂₋₁₀ alkenyl optionally containing one or more non-adjacent heteroatoms selected from N, O, and S, and optionally substituted with a carbocyclic or heterocyclic ring; or R² is an optionally substituted heterocyclic ring, aryl or heteroaryl;

R⁵ is a substituent at any position on W; and is H, OR², C₁₋₆ alkyl, C₂₋₆ alkenyl, each optionally substituted by halo, ═O or one or more heteroatoms; or R⁵ is an inorganic substituent; and

W is an optionally substituted aryl or heteroaryl, which may be monocyclic or fused with a single or multiple ring and optionally containing a heteroatom;

or a compound having formula (TA1-2):

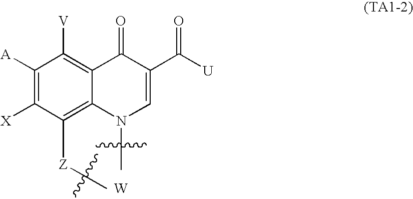

and pharmaceutically acceptable salts, esters and prodrugs thereof;

wherein V, A, X, Z and U are as defined in formula TA1-1, and W is selected from the group consisting of

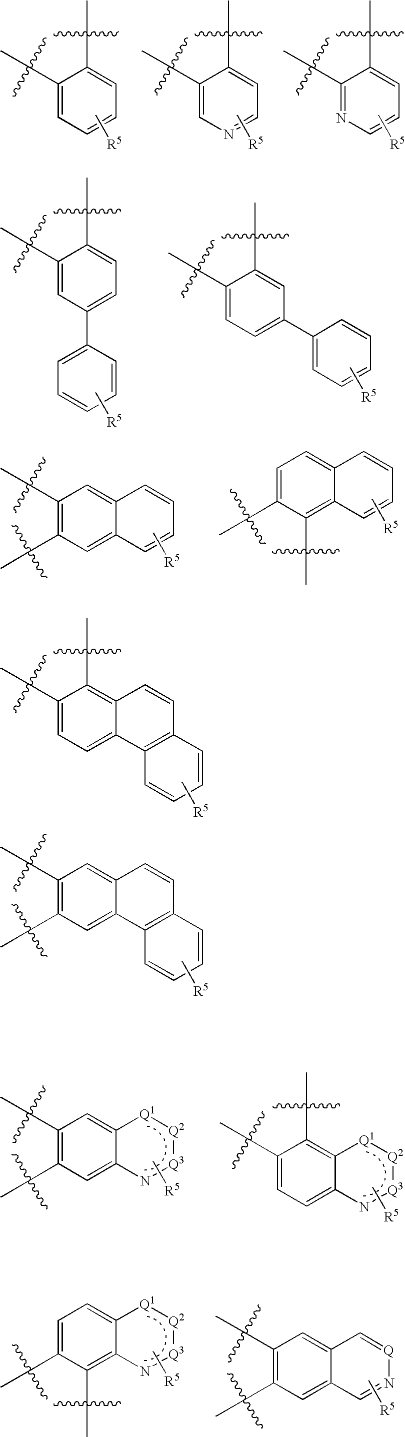

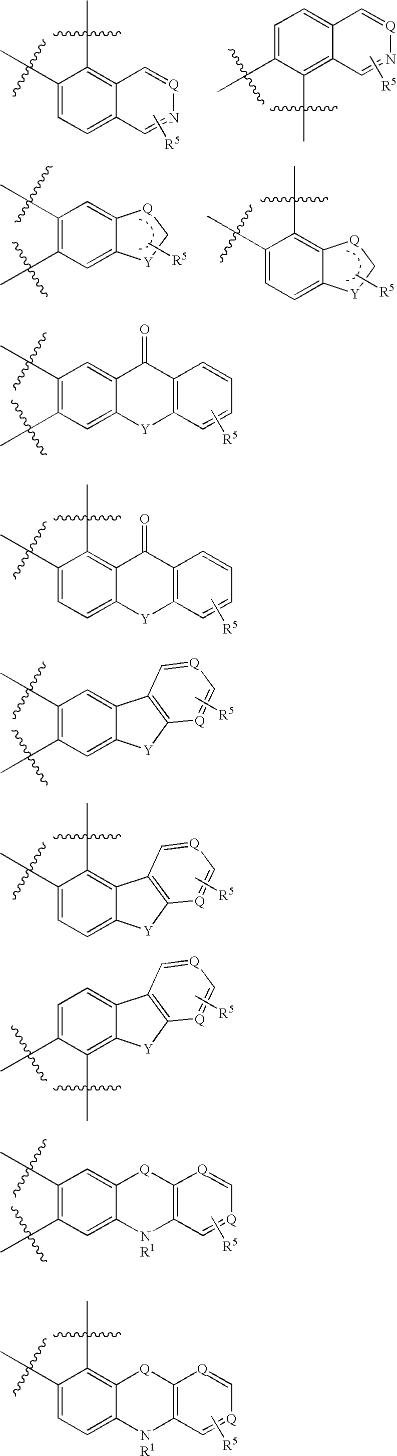

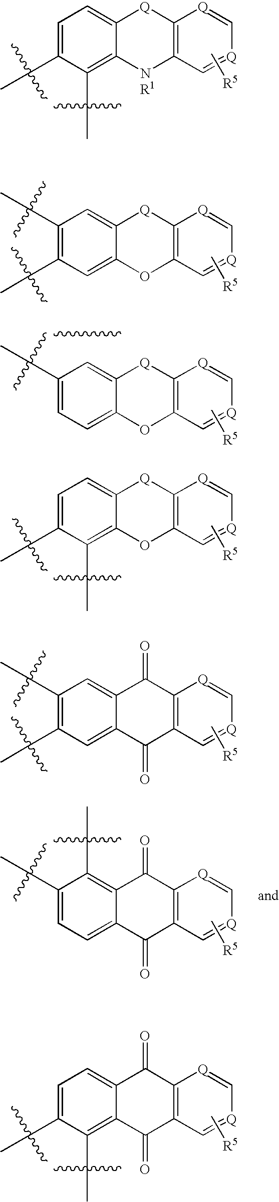

wherein Q, Q¹, Q², and Q³ are independently CH or N;

Y is independently O, CH, ═O or NR¹; and

R⁵ is as defined in formula TA1-1;

whereby said disorder is treated.

C19. The method of embodiment C18, wherein said disorder is a cancer selected from the group consisting of cancers of the colorectum, breast, ovary, lung, thymus, liver, pancreas, lymph node, stomach, appendix, small bowel, colon, rectum, prostate, brain, head and neck, skin, kidney, heart, adrenal, pituitary, parathyroid, thyroid, bone marrow and blood.

C20. The method of embodiment C18 or C19, wherein said disorder is a cancer of the bone marrow or blood.

C21. The method of embodiment C18, C19 or C20, wherein the compound has the formula:

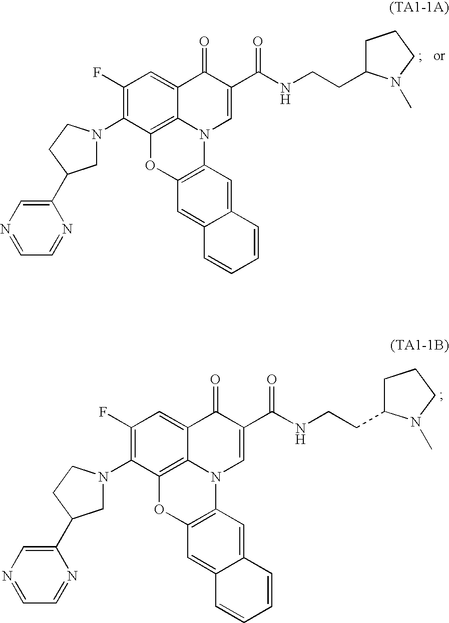

or a pharmaceutically acceptable salt, ester or prodrug thereof, or a specific isomer or mixture of isomers thereof.

C22. The method of embodiment C21, wherein the compound has the formula (TA1-1B), or a pharmaceutically acceptable salt thereof.

Citation of patents, patent applications, publications and documents herein is not an admission that any of the foregoing is pertinent prior art, nor does it constitute any admission as to the contents or date of these publications or documents.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following aspects.

What is claimed is:

1. A method for inhibiting proliferation of cancer cells, comprising contacting a cancer cell with an effective amount of a compound having the formula: 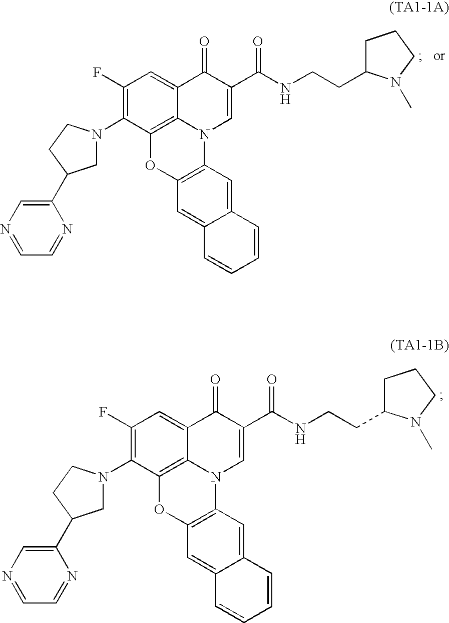
or a pharmaceutically acceptable salt, ester or prodrug thereof, or a specific isomer or mixture of isomers thereof.

2. The method of claim 1, wherein said cancer cells are selected from the group consisting of leukemia cells, lymphoma cells, breast cancer cells, lung cancer cells, central nervous system cancer cells, skin cancer cells, ovarian cancer cells, prostate cancer cells, renal cancer cells, colorectal cancer cells, liver cancer cells, pancreatic cancer cells, adrenal gland cancer cells, thymic cancer cells, lymph node cancer cells, stomach cancer cells, appendix cancer cells, small bowel cancer cells, head and neck cancer cells, heart cancer cells, pituitary gland cancer cells, parathyroid gland cancer cells, and thyroid gland cancer cells.

3. The method of claim 2, wherein said cancer cells are central nervous system cancer cells.

4. The method of claim 3, wherein said central nervous system cancer cells are brain cancer cells.

5. The method of claim 4, wherein said brain cancer cells are gliomablastoma cells or medulloblastoma cells.

6. The method of claim 4, wherein the compound has the formula (TA1-1B), or a pharmaceutically acceptable salt thereof.

7. The method of claim 2, wherein said cancer cells are leukemia cells.

8. The method of claim 7, wherein said leukemia cells are chronic lymphocytic leukemia (CLL) cells and said compound has the formula (TA1-1B), or a pharmaceutically acceptable salt thereof.

9. The method of claim 1, wherein said cancer cells are cultured in vitro.

10. A method for inhibiting proliferation of a tumor overexpressing one or more peptide receptors, comprising administering to a subject in need thereof a therapeutically effective amount of a compound having formula (TA1-1): 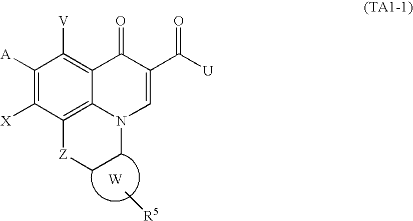
and pharmaceutically acceptable salts, esters and prodrugs thereof;
wherein V is H, halo, or NR¹R²;
A is H, fluoro, or NR¹₂;
Z is O, S, NR¹ or CH₂;
U is OR² or NR¹R²;
X is OR², NR¹R², halo, azido, or SR²;
n is 1-3;
wherein in NR¹R², R¹ and R² may form a double bond or a ring, each of which is optionally substituted;
R¹ is H or a C₁₋₆ alkyl;
R² is H or a C₁₋₁₀ alkyl or C₂₋₁₀ alkenyl optionally containing one or more non-adjacent heteroatoms selected from N, O, and S, and optionally substituted with an optionally substituted carbocyclic or heterocyclic ring; or R² is an optionally substituted heterocyclic ring, aryl or heteroaryl;
R⁵ is a substituent at any position on W; and is H, OR², C₁₋₆ alkyl, C₂₋₆ alkenyl, each optionally substituted by halo, ═O or one or more heteroatoms; or R⁵ is an inorganic substituent; and
W is an optionally substituted aryl or heteroaryl, which may be monocyclic or fused with a single or multiple ring and optionally containing a heteroatom;
or a compound having formula (TA1-2): 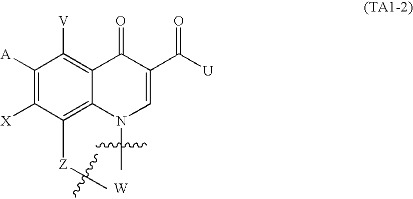
and pharmaceutically acceptable salts, esters and prodrugs thereof;
wherein V, A, X, Z and U are as defined in formula TA1-1, and W is selected from the group consisting of 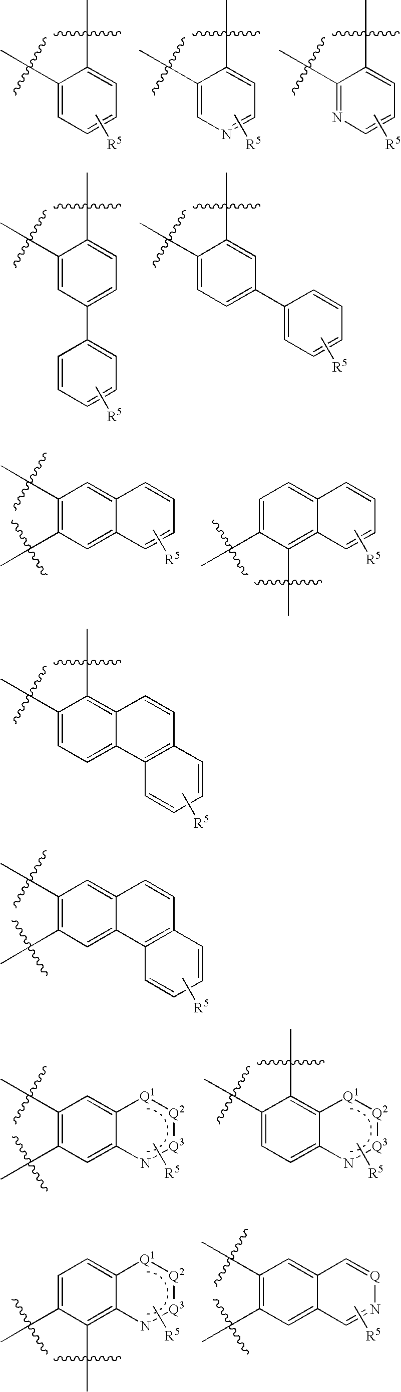 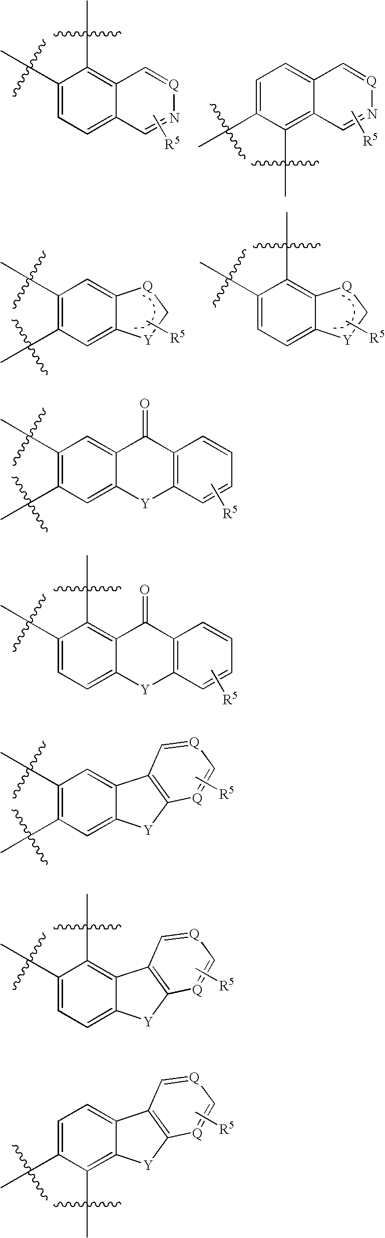 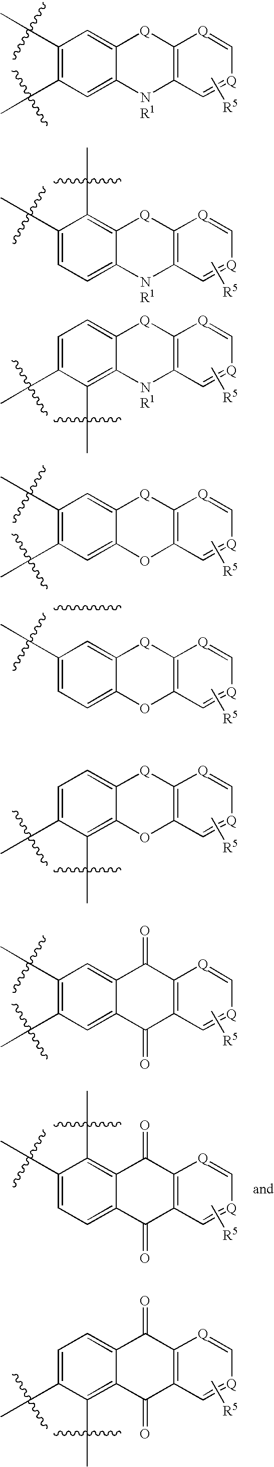
wherein Q, Q¹, Q², and Q³ are independently CH or N;
Y is independently O, CH, ═O or NR¹; and
R⁵ is as defined in formula (TA1-1);
whereby tumor proliferation is inhibited.

11. The method of claim 10, wherein said peptide receptor is selected from the group consisting of the somatostatin receptors sst1-sst5, the VIP receptors VPAC1 and VPAC2, the CCK1 and CCK2 receptors, the bombesin receptor subtypes BB 1(NMB receptor), BB2 (GRP receptor) and BB3, and GLP-1 receptors.

12. The method of claim 10, wherein said tumor is a neuroendocrine tumor selected from the group consisting of multiple endocrine neoplasia type 1 (MEN1), multiple endocrine neoplasia type 2 (MEN2), carcinoid tumors, islet cell tumors, pheochromocytomas and paragangliomas.

13. The method of claim 10, wherein said tumor is a neuroendocrine tumor selected from the group consisting of medullary thyroid carcinomas, small-cell lung cancers, gastrointestinal stromal tumors (GIST), gastroenteropancreatic tumors (GEP NETs), paragangliomas, pheochromocytomas, exocrine pancreatic cancers, Ewing's sarcomas, adrenal tumors, growth hormone pituitary adenomas, nonfunctioning pituitary adenomas, parathyroid adenomas, gastrinomas, glucagonomas, insulinomas, VIPomas, adrenal tumors, gut carcinoids, ileal carcinoids, and bronchial carcinoids.

14. The method of claim 10, wherein said tumor is a tumor of the central nervous system selected from the group consisting of astrocytomas, meningiomas, schwannomas, medulloblastomas and glioblastomas.

15. The method of claim 10, wherein said tumor is a tumor of the reproductive system selected from the group consisting of breast carcinomas, endometrial carcinomas, leiomyomas, ovarian cancers, epithelial and stromal tumors, and prostate carcinomas.

16. The method of claim 10, wherein the compound has the formula: 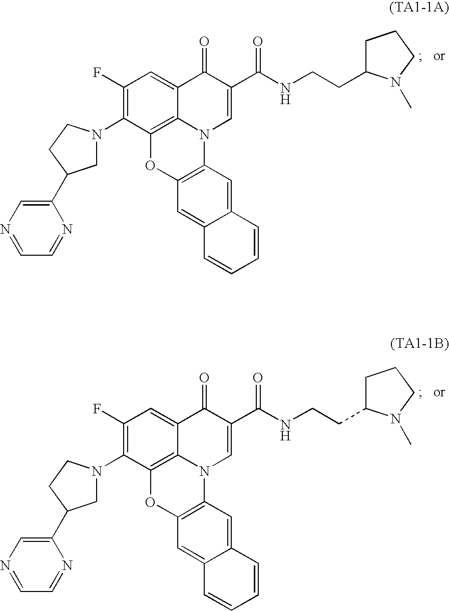
or a pharmaceutically acceptable salt, ester or prodrug thereof, or a specific isomer or mixture of isomers thereof.

17. The method of claim 16, wherein the compound has the formula (TA1-1B), or a pharmaceutically acceptable salt thereof.

18. A method for treating a disorder resulting from aberrant cell proliferation, comprising administering to a subject in need thereof a therapeutically effective amount of a compound having formula (TA1-1): 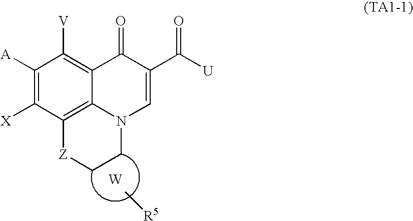
and pharmaceutically acceptable salts, esters and prodrugs thereof;
wherein V is H, halo, or NR¹R²;
A is H, fluoro, or NR¹₂;
Z is O, S, NR¹ or CH₂;
U is OR² or NR¹R²;
X is OR², NR¹R², halo, azido, or SR²;
n is 1-3;
wherein in NR¹R², R¹ and R² may form a double bond or a ring, each of which is optionally substituted;
R¹ is H or a C₁₋₆ alkyl;
R² is H or a C₁₋₁₀ alkyl or C₂₋₁₀ alkenyl optionally containing one or more non-adjacent heteroatoms selected from N, O, and S, and optionally substituted with an optionally substituted carbocyclic or heterocyclic ring; or R² is an optionally substituted heterocyclic ring, aryl or heteroaryl;
R⁵ is a substituent at any position on W; and is H, OR², C₁₋₆ alkyl, C₂₋₆ alkenyl, each optionally substituted by halo, ═O or one or more heteroatoms; or R⁵ is an inorganic substituent; and
W is an optionally substituted aryl or heteroaryl, which may be monocyclic or fused with a single or multiple ring and optionally containing a heteroatom;
or a compound having formula (TA1-2): 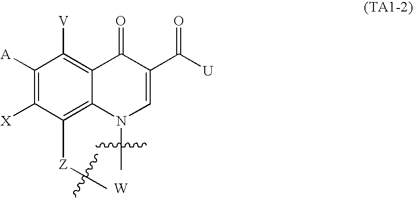
and pharmaceutically acceptable salts, esters and prodrugs thereof;
wherein V, A, X, Z and U are as defined in formula TA1-1, and W is selected from the group consisting of 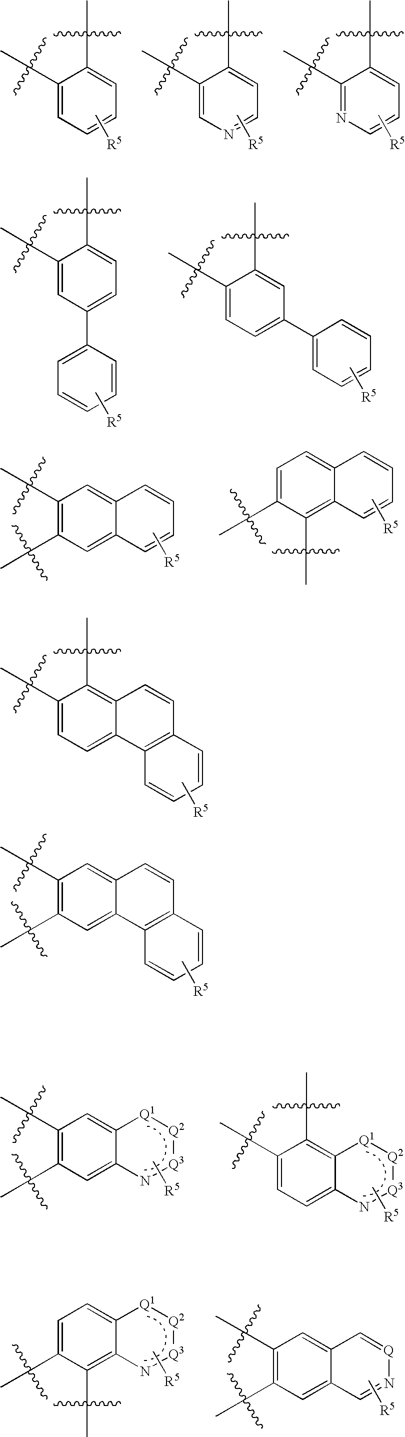 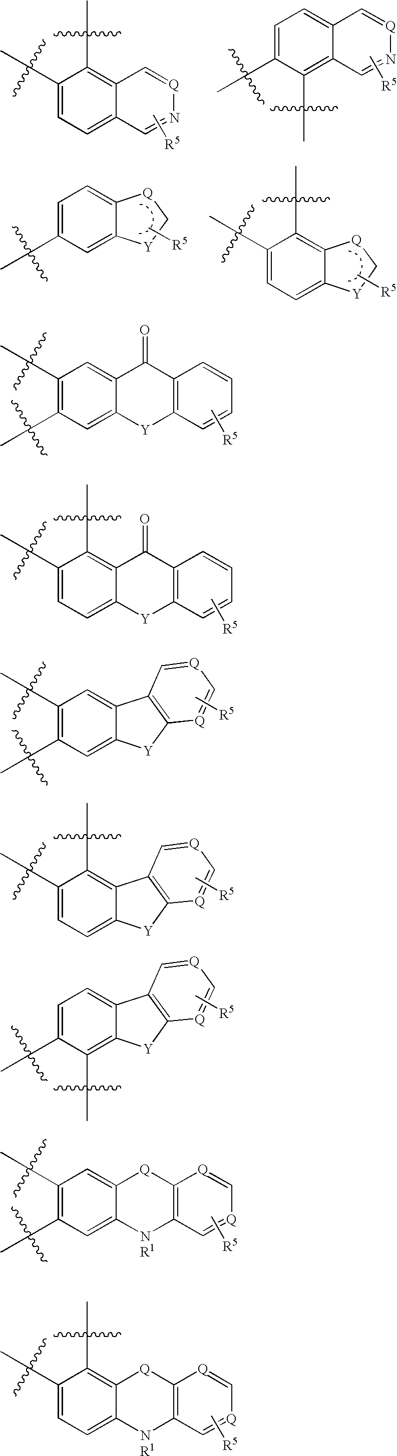 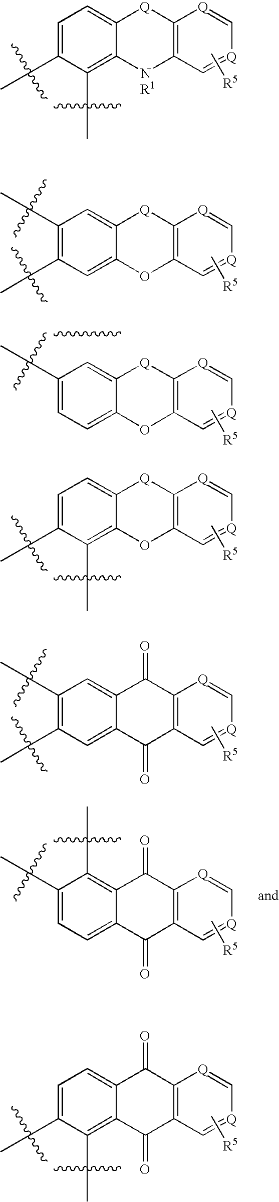
wherein Q, Q¹, Q², and Q³ are independently CH or N;
Y is independently O, CH, ═O or NR¹; and
R⁵ is as defined in formula TA1-1;
whereby said disorder is treated.

19. The method of claim 18, wherein said disorder is a cancer selected from the group consisting of cancers of the colorectum, breast, ovary, lung, thymus, liver, pancreas, lymph node, stomach, appendix, small bowel, colon, rectum, prostate, brain, head and neck, skin, kidney, heart, adrenal, pituitary, parathyroid, thyroid, bone marrow and blood.

20. The method of claim 18, wherein said disorder is a cancer of the bone marrow or blood.

21. The method of claim 18, wherein the compound has the formula: 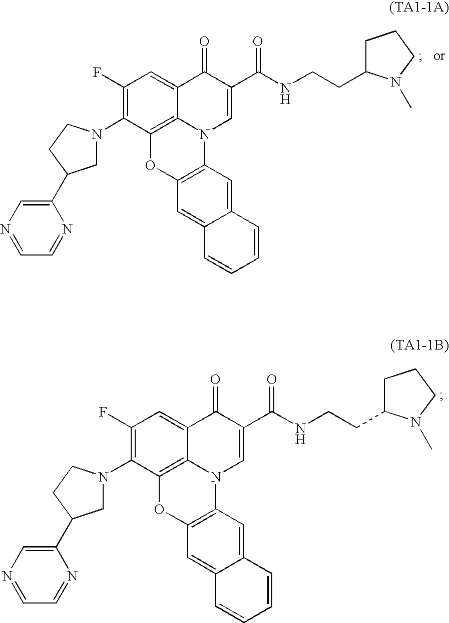
or a pharmaceutically acceptable salt, ester or prodrug thereof, or a specific isomer or mixture of isomers thereof.

22. The method of claim 21, wherein the compound has the formula (TA1-1B), or a pharmaceutically acceptable salt thereof.
